(19) United States
(12) Patent Application Publication
(10) Pub. No.: US 2020/0253547 A1
(43) Pub. Date: Aug. 13, 2020

(54) Monitoring System for Assessing Control of a Disease State

(71) Applicant: Apple Inc., Cupertino, CA (US)

(72) Inventors: Bronwyn Harris, Redwood City, CA (US); Todd Murphy, Mountain View, CA (US); Michael Carchia, San Francisco, CA (US)

(21) Appl. No.: 16/757,125

(22) PCT Filed: Oct. 31, 2018
(86) PCT No.: PCT/US2018/058558

§ 371 Date: Apr. 17, 2020

Related U.S. Application Data
(60) Provisional application No. 62/579,794, filed on Oct. 31, 2017.
(60) Provisional application No. 62/649,448, filed on Mar. 28, 2018.

(51) Int. Cl. A61B 5/00; A61B 5/024; A61B 5/08; A61B 5/11; G16H 10/60

(52) U.S. Cl. CPC A61B 5/4842; A61B 5/7275; A61B 5/746; A61B 5/0022; A61B 5/02405; A61B 5/0816; G16H 10/60; A61B 5/4806; A61B 5/7203; A61B 5/7465; A61B 5/7221; A61B 5/6887; A61B 5/1102

(57) ABSTRACT

Devices, systems and methods are provided to assist with the monitoring or management of a patient's medical condition, which have one or more sensors sensing individual patient data on or near the patient. This individual patient data corresponds to at least one physiological parameter of the patient and includes a sensor that does not require the patient to apply it or activate it. The data is then transmitted to a processor for computing a risk or status signal that is based on comparison from a baseline related to a patient or related population and an alert or alarm can be generated based on the result of the signal.

### CROSS-REFERENCE TO RELATED APPLICATION

This application is a national phase application under 35 U.S.C. § 371 of International Patent Application PCT/US2018/058558, filed Oct. 31, 2018, which claims priority to U.S. Provisional Patent Application No. 62/579,794 filed Oct. 31, 2017, and U.S. Provisional Patent Application No. 62/649,448 filed Mar. 28, 2018, the contents of which are incorporated herein by reference as if fully disclosed herein.

This application is related to noninvasive, diagnostic, medical devices, systems and methods. More specifically, this application is related to methods for monitoring and managing chronic medical conditions.

### BACKGROUND

Management of chronic diseases is a large and costly problem in the United States accounting for over 80% of the nation's healthcare costs and for seven out of every ten deaths.

Asthma is one example of a chronic disease that takes a significant toll on individual patients and the healthcare system as a whole. Asthma is a chronic inflammatory disease of the airways, characterized by variable and recurring symptoms. Periods of acute worsening in symptoms, known as exacerbations, are a major feature of the disease. Approximately 300 million individuals worldwide are affected by asthma, and in the United States there are approximately 23 million individuals with asthma, resulting in 1.7 million emergency department visits each year and 10.1 million lost workdays.

Asthma is one of the most common chronic diseases in children, affecting 7.1 million children in the United States alone. If not managed properly, it can be life-threatening, with over 3,000 deaths due to asthma among children under the age of 15 years in the United States in 2011. Apart from being life-threatening, asthma also has a significant impact on morbidity and quality of life in children and their families. Asthma is the leading cause of school absenteeism. In 2008, for example, an estimated 14 million lost school days were attributed to asthma, and children with persistent asthma have been shown to perform lower on standardized testing.

Asthma is costly to the healthcare system, totaling an estimated 56 billion dollars in annual healthcare expenditures in the United States alone. A disproportionate amount of this spending can be attributed to poor disease control. According to Aetna, a major health insurance provider, an emergency department visit for a pediatric asthmatic patient can cost the insurer $600 and a hospitalization can cost $6,600. A person's asthma can be classified in various ways. Using one common classification scheme, there are 2.9 million children in the United States with moderate to severe persistent asthma. A patient with moderate asthma has a 3% chance of being hospitalized and an 11% chance of going to the emergency department in a three-month period. A patient with severe asthma has a 10% chance of being hospitalized and a 21% chance of going to the emergency department in a three-month period.

Some treatments and management strategies currently exist for asthma. For example, written asthma action plans have been demonstrated to be a relatively effective tool for some patients in improving control over asthma symptoms. Use of these tools is becoming more frequent, and increasing the proportion of persons with asthma who use these plans is part of the U.S. Department of Health and Human Services' Healthy People 2020 goals. Typically, these written plans help individuals and families self-manage their illness by guiding their use of various environmental modifications or medical treatments available (e.g., inhalers, oral steroids) and when to contact their healthcare providers. These action plans, however, use relatively subjective criteria to define exacerbations, and many of the signs, such as tachypnea (abnormally rapid breathing) and nighttime cough frequency, are late findings and/or are difficult to measure. Some asthmatics also use peak flow meters—small devices into which the patient blows in order to measure lung function. Although the meters provide quantitative and objective measurements, the results obtained are effort-dependent and require longitudinal daily measurements. For this reason, the results obtained are highly variable, particularly in children.

Another tool to help manage asthma, in particular to assess the control of the patient's asthma and adjust controller medications accordingly, is the asthma control test. Similar to the asthma action plan, however, the asthma control test relies on the families of asthma patients to assess and report symptoms accurately and routinely.

A number of technologies have been developed to allow asthma patients to better monitor their disease outside the hospital. These technologies mainly target improving adherence to therapy or detection of exacerbations. One challenge of some of these technologies, however, is that they use relatively late indicators of worsening disease status that limit their ability to improve the effectiveness of short-term and long-term disease management. Other technologies focus on trying to reduce exacerbation events, which represents only a small component of what it means to have control over a disease. These technologies do not provide insights into long-term control and are not developed as a management tool for clinicians that would inform pharmacologic therapy choices and other interventions designed to improve long-term control.

Therefore, a substantial gap remains in the ability to reliably measure and monitor asthmatic status outside of healthcare facilities. It would thus be desirable to have a system and method for monitoring asthma status outside the hospital, which would empower families and healthcare providers to more effectively manage the disease. Ideally, such a system and method would help provide improved disease monitoring and management, relative to currently available systems and methods. Also ideally, the system and method might be used for monitoring other disease states, such as allergies, chronic obstructive pulmonary disease, diabetes, hypertension, autoimmune disorders, migraine or other neurologic disease, obstructive sleep apnea, cystic fibrosis, arthritis and other rheumatologic conditions, seizure disorders, cardiovascular disease, peripheral vascular disease and/or congestive heart failure. Common patient monitoring systems measure a variable, for example heart rate or blood oxygenation and alert or alarm if a simple threshold is breached. Prior work has described using parameters obtained while a patient is sleeping and instituting simple cut-offs or thresholds to define changes in control and potentially trigger alerts. However utilizing these simple cut-offs either results in too frequent false alarms or very high thresholds, which result in missing relevant early warning signs. The embodiments described below attempt to better utilize this data to improve disease control assessment and tie it to useful interventions.

### SUMMARY

Devices, systems and methods are provided to assist with the monitoring or management of a patient's medical condition, which has one or more sensors sensing individual patient data on or near the patient. This individual patient data corresponds to at least one physiological parameter of the patient and includes a sensor that does not require the patient to apply it or activate it. The data is then transmitted to a processor for computing a risk or status signal that is based on comparison from a baseline related to a patient or related population and an alert or alarm can be generated based on the result of the signal.

In one embodiment, a system to assist in the controlling of a patient's medical condition is provided, comprising one or more sensors configured to sense individual patient data on or near a patient, wherein the individual patient data is related to at least one physiological parameter of the patient, wherein at least one of the sensors comprises a passive sensor that does not require patient activation or patient contact, a transmitter configured to transmit the individual patient data to a processor, and a processor configured to calculate a signal using the individual patient data and at least one of a baseline patient data or population data, wherein the processor is further configured to calculate the signal using a combination of a proportional change, integral change and derivative change of the individual patient data and the at least one of the baseline patient data or population data. The individual patient data may be derived patient data. The processor may be further configured to generate the combination of the proportional change, integral change and derivative change from a combination of individual and non-individual data. The combination of individual and non-individual data utilizes a combination including at least two of the following: physiologic, home, local environment, or medical health records. The processor may be further configured to generate an alert to the patient or a care provider of the patient. The alert may include at least one of a medication change, avoidance of regional trigger, behavioral change, environmental change, or education. The system may also further comprise at least two sensors and wherein the processor is further configured to distinguish individual patient between different people in a bed. The processor may also be further configured to receive a response from the patient or a caregiver and to automatically adjust the status signal. The processor may also be further configured to calculate an inspiratory time, an expiratory time, and/or an inspiratory/expiratory (I/E) ratio using the one or more sensors.

In another embodiment, a method to assist in the controlling of a patient's medical condition is provided, comprising using one or more sensors configured to sense individual patient data on or near a patient, wherein the individual patient data is related to at least one physiological parameter of the patient, wherein at least one of the sensors comprises a passive sensor that does not require patient activation or patient contact, transmitting the individual patient data to a processor, and calculating a signal with the processor using the individual patient data and at least one of a baseline patient data and population data; and using a combination of a proportional change, integral change and derivative change in the individual patient data and the at least one of the baseline patient data and population data. The individual patient data may be derived individual patient data. The baseline patient data may be generated from patient medical health records. Calculating the signal further may optionally utilize home or local environment data, if available. The system may also generate an alert to the patient or a care provider of the patient. The alerts may include at least one of medication changes, an avoidance of regional trigger, a behavioral change, an environmental change, and patient education information. The method may optionally comprise calculating at least one of an inspiratory duration, an expiratory duration, and an I/E ratio using the one or more sensors, and comparing the at least one of the inspiratory duration, the expiratory duration, and the I/E ratio to a corresponding the inspiratory duration, the expiratory duration, and the I/E ratio to a corresponding threshold value. The corresponding threshold value may be an absolute value, and/or a patient-specific value derived from the at least one physiological parameter. The method may further comprise calculating the I/E ratio from a signal received from the passive sensor. The at least one of the inspiratory duration, the expiratory duration, and the I/E ratio may be calculated using beat-to-beat variations in the heart activity detected by the one or more sensors.

### BRIEF DESCRIPTION OF THE DRAWINGS

FIG. 1 is a flow diagram illustrating a method for receiving and processing patient information to provide a personalized report, according to one embodiment.

FIG. 2 is a schematic illustration of a system for receiving and processing patient information to provide a personalized report, according to one embodiment.

FIG. 3 is a flow diagram illustrating part of a method for using various patient information to provide a personalized report and generate a feedback loop, according to one embodiment.

FIG. 4 is a flow diagram illustrating a method aggregating data from sensors and non-sensor sources to enhance outcomes, according to one embodiment.

FIG. 5 is a side view figurative representation of a patient lying on a bed with a below-mattress sensor.

FIG. 6 is a side view figurative representation of a patient lying on a bed with a below-mattress sensor.

FIG. 7 is a figurative representation of a system for receiving and processing patient information to provide a personalized report, according to one embodiment.

FIG. 8 is a flow diagram illustrating an exemplary method for assisting control of a patient's medical condition.

FIG. 9 is an exemplary table depicting signals with corresponding messaging and colors.

FIGS. 10A and 10B are exemplary state diagrams of the alert interactions with a user.

FIG. 11 depicts an exemplary software workflow for processing data.

FIG. 12 depicts another exemplary software workflow.

FIG. 13 depicts multiple out of control conditions based on lower control limit and upper control limit.

FIG. 14 depicts a graph of a patient's data showing the product of the standard deviation of heart rate and the standard deviation of the respiratory rate over time.

FIG. 15 depicts a graph of the same patient data shown in FIG. 14, but as the individual heart rate and respiratory rate (50^{th} percentile for the night) over time.

FIG. 16 depicts a few drivers of asthma and also signs and symptoms.

FIG. 17 is one potential embodiment of how this collected data may be analyzed and then used as part of a system to improve asthma management.

FIG. 18 depicts a potential way that the output of the bed sensor may be used to calculate I/E ratio and examples of how it may be used in distinguishing asthma related events from non-asthma related events and among the asthma related events, the severity of symptoms.

FIG. 19 depicts a normal breathing pattern. The respiratory amplitude is shown on the y-axis with time on the x-axis.

FIG. 20 is an example of a spirogram which shows an idealized volume vs time curve.

FIG. 21 is an example of a spirogram, which shows a volume vs time curve for a prolonged I/E ratio.

FIG. 22 depicts changes in capacitance during respiratory cycle.

FIG. 23 depicts a typical Cheyne-Stokes breathing pattern.

FIG. 24 depicts a respiratory signal using a capacitance sensor, showing a change in breathing pattern from normal respiration to sniffing.

FIG. 25 is an exemplary graphical user interface (GUI) displaying a dashboard highlighting high-level patient status and other information for a user.

FIG. 26 is an exemplary GUI displaying an alerts menu providing a listing of alerts applicable to the patient.

FIG. 27 is an exemplary GUI displaying a tracking menu including a navigable calendar view.

FIG. 28 is an exemplary GUI displaying a user interface for logging patient symptoms.

FIG. 29 is an exemplary GUI displaying a tracking menu including a navigable list view.

FIG. 30 is an exemplary GUI displaying a tracking menu communicating risk score value.

FIG. 31 is an exemplary GUI 3100 displaying a learning modules menu.

FIG. 32 is an exemplary GUI 3200 displaying a tools menu.

FIGS. 33A-33H are a set of exemplary GUIs as part of an interactive user workflow in which the user is prompted to provide specific health-related information relating to an alert.

FIGS. 34A-34C are a set of exemplary GUIs forming part of an interactive user workflow in which the user is provided with personalized educational information to improve control of asthma conditions.

FIGS. 35A-35E are another set of exemplary GUIs forming part of an interactive user workflow in which the user is provided with personalized educational information to improve control of asthma conditions.

FIG. 36 is a plot displaying exemplary patient data including various derived parameters over time.

FIG. 37A is a plot displaying exemplary patient data including heart rate and respiratory rates over time.

FIG. 37B is a plot displaying corresponding exemplary risk score values for a patient over time.

FIG. 38A is a schematic of a normal heart rate pattern for a healthy individual.

FIG. 38B is a schematic of a heart rate pattern for an unhealthy individual.

### DETAILED DESCRIPTION

The following description of various embodiments should not be interpreted as limiting the scope of the invention as it is set forth in the claims. Other examples, features, aspects, and advantages may be included in various embodiments, without departing from the scope of the invention. Additionally, some of the descriptions below focus on systems and methods for monitoring asthma. In alternative embodiments, however, the systems and methods described herein may be used (or modified for use) for monitoring any of a number of different disease states, such as but not limited to allergies, chronic obstructive pulmonary disease, diabetes, hypertension, autoimmune disorders, migraine or other neurologic disease, obstructive sleep apnea, cystic fibrosis, arthritis and other rheumatologic conditions, seizure disorders, cardiovascular disease, peripheral vascular disease and/or congestive heart failure. Accordingly, the drawings and descriptions should be regarded as illustrative in nature and not restrictive.

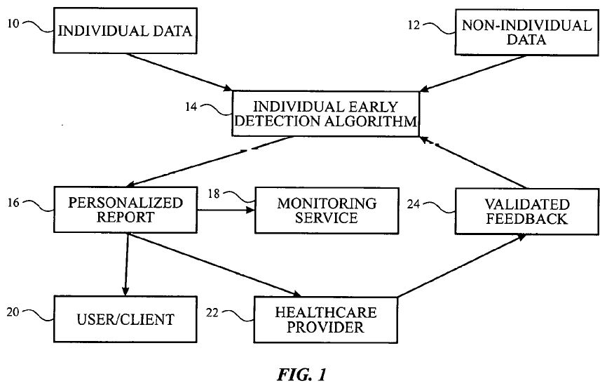

Generally, referring to FIG. 1, a method for monitoring a patient's chronic disease state, according to one embodiment, is illustrated in a flow diagram. This method may be used for monitoring the disease state in one patient or multiple patients, according to various alternative embodiments. In some embodiments, the method may be used for early detection of changes in the disease state. As mentioned previously, asthma is one example of a disease that may be monitored using this method.

A patient may be defined as an individual with a chronic disease who is being directly monitored and assessed. The user who receives the data may be the patient, or a caregiver (e.g., a parent of a patient who is a child, a member of a medical team or a member of a third party, etc.).

As a first step in the method, individual patient data 10 and non-individual patient data 12 are received in a computer processor 14. In alternative embodiments, the processor may receive only individual data 10 or only non-individual data 12. Individual patient data 10 may include, for example, physiological data, past medical, family, and/or surgical history, and/or environmental data pertaining to an environment around or near the patient. A patient monitoring system, which is described further below, may be designed to collect any of a number of different types of individual data 10 from the patient. This individual data 10 may be collected using one or more devices that contact the patient and/or that collect data without contacting the patient (“contactless” devices). Types of individual data 10 that may be collected include, but are not limited to, respiratory rate and/or effort, heart rate and/or variability, oxygen saturation, weight, movement, sleep quality (including number of full or partial awakenings and time of daily final awakening over time), body temperature, exhaled breath composition (including but not limited to carbon dioxide, nitrogen, carbon monoxide), voice pitch and/or other sound-based parameters, air quality, humidity, and/or other environmental data. Examples of non-individual data 12 include, but are not limited to, regional environmental data, such as demographic data and aggregate population data.

After the individual data 10 and non-individual data 12 are received by the processor 14, the data are processed to generate an individual, personalized patient report 16, which may be used to assess status of the patient's disease, such as asthma, hypertension, chronic obstructive pulmonary disease, diabetes, mental health disease, and/or other chronic conditions. This personalized report 16 may be transmitted automatically to a monitoring service 18, to the patient and/or other user 20, and/or to a healthcare provider 22. The healthcare provider 22 (or other user) may then provide validated feedback 24, which may feed back into the processor for further refinement. The processor may generate a patient-specific model, which uses feedback 24 from the health care provider and/or user, regarding symptoms and acute clinical exacerbations to train and improve the model. In this way, the model may be personalized to an individual patient. Using the personalized model, the processor 14 may provide each patient with the personalized report 16 and in some cases multiple reports over time, summarizing the current status of the patient's disease state and/or the status over a period of time. The process of providing feedback to the processor/model, making the model more personalized to an individual patient, and providing another, more personalized report may be repeated as many times as desired for a patient over a course of time. Again, FIG. 1 illustrates only one, exemplary embodiment of the monitoring method. Alternative embodiments may include fewer or additional steps.

In some embodiments of an asthma monitoring method, for example, one or more sensors located under a patient's mattress are used to collect heart rate and respiratory rate individual patient data 10 from the patient (also referred to as “input data”). This individual data 10 is transmitted to the system's processor, which generates a personalized model or uses a preexisting model. In some embodiments, the processor may reside in the cloud or other remote location. An example of a personalized model is one that analyzes the input data against the patient's baseline data (e.g., normal heart rate and respiratory rate for that patient). The processor, using the model, will generate a personalized report 16, showing the patient's current disease state relative to a normal state for the patient. For example, the report could indicate that the patient's heart rate for the past two nights has been 10% above her baseline heart rate. In some embodiments, the system may be configured to generate any of a number of alerts or recommendations, which may be transmitted to the patient, healthcare provider and/or other user. For example, the system may generate a text alert if the patient's heart rate has been 20% above her baseline heart rate for the past two nights. In some embodiments, the alert may inform the patient (and/or others) as to the patient's disease condition and may also provide a recommendation, such as that the patient should contact his/her healthcare provider for further diagnosis.

The hardware may be a disc-like device that is placed under the mattress below where the user sleeps and can measure vibrations from the user, which can be translated into physiologic data, sleep parameters, movement and more about the user. In other embodiment, the device may be a different, flattened-shape, such as a square, rectangle, oval, or other shape. An alternative hardware may be a thin strip of material that is placed on top of the mattress, but under the bed sheet. The user will lie directly on top of this sensor. Yet another alternative may be a small accelerometer based sensor that can be placed on the top, side or under the mattress and does not need to be in close proximity to the user.

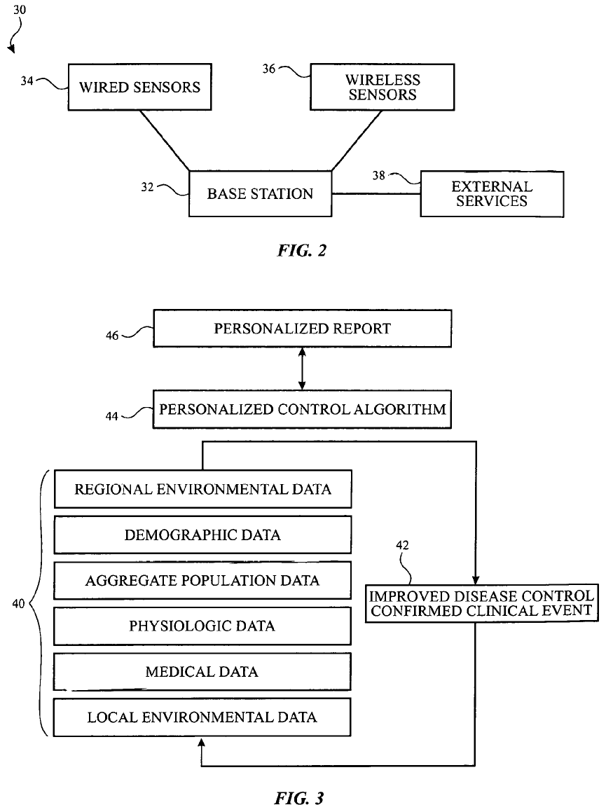

Referring now to FIG. 2, one embodiment of a disease state monitoring system 30 is illustrated, which may be used to generate the individual patient data 10 used in the method described above. In this embodiment, the system 30 includes a base station 32, one or more wired sensors 34, one or more wireless sensors 36 and one or more external services 38. In alternative embodiments, the system 30 may include only wired sensors 34, only wireless sensors 36, only external services 38, any combination thereof, or any other combination of sensors and/or services. The base station 32 may include a local storage mechanism, a processor, and a connector to a remote server. This connector may be a wired connection, such as a USB or CAT6 network connection, or may be a wireless communication that utilizes WiFi in the home, or Bluetooth to a device such as a mobile phone, which can then connect the data to a remote server utilizing WiFi or cellular data. In alternative embodiments, the base station 32 may not include a processor and may simply act as a receiver of data from one or more sensors or other sources and a transmitter of that data to a separate processor, such as a processor located in the cloud or other remote location. In some embodiments, the base station 32 may also be configured to store sensor data after it is received. In some embodiments, the processor in the base station 32 may also pre-process the received data before transmitting the data to a remote server for further processing. Pre-processing the data may include, but is not limited to, compressing the data, extracting features from the data, and/or generating a local model of the data. The base station 32 can also act as a node in a distributed computing platform, which may be necessary for cost-effective computation of extensive datasets. Raw data can be sent to this node via the remote server or a peer-to-peer network and can then be processed using the computing power of this node.

The sensors 34, 36 may be connected to the base station 32 either through a wired (including electrically connected) or wireless connection, including, but not limited to, RFID, Bluetooth, WiFi, and/or wireless languages that allow everyday devices to connect. The sensors 34, 36 can be within the base station 32 enclosure or outside the enclosure. The sensors 34, 36 can communicate with the base station 32 either in real-time or stored locally and then transferred later through a wired or wireless connection. The sensors 34, 36 themselves may optionally include a processor and/or local data storage. Such processor and storage could, in some embodiments, serve the functions of the base station 32 and thereby eliminate the need for a separate base station.

One or more external services 38 may also provide data to the base station 32. For example, these services may include sensors connected to home thermostats, smart home sensing systems, or regional meteorological measuring equipment that are able to provide data as external services. Likewise, the base station 32 can also communicate with other external services 38, for example, such as services that can control temperature, humidity, or air purification.

With reference now to FIG. 3, one embodiment of a data flow and analysis is shown in greater detail. In this embodiment, data is received into the system from multiple sources 40, such as but not limited to physiologic data related to the individual or group of individuals, local environmental data from where the individual spends a substantial amount of time (for example, home, work, vehicle), geographic environmental data from sources such as those found available online, and/or clinical symptom data obtained from an individual or group of individuals. The data available from these sources are processed 44 for each individual or group of individuals, and feedback regarding disease state and control is provided, such as in the form of a report 46, to the individual, group of individuals, and/or healthcare provider(s). Improved disease control and confirmed clinical events (e.g., worsening of disease state requiring treatment alteration, clinic visit, emergency care or hospitalization, etc.) may also be recorded 42 and will be used to improve the personalized algorithm to determine disease control. The system, with or without human interaction, continuously monitors each individual for changes to his/her disease state. If the estimated condition has worsened beyond a calculated level based on individual or population-level historical data analysis, the individual, group of individuals, guardian and/or the healthcare provider may be notified via text messaging, email, phone call, videoconference, personal visit, integration with the electronic medical record, combinations of any of these methods and/or any other suitable methods.

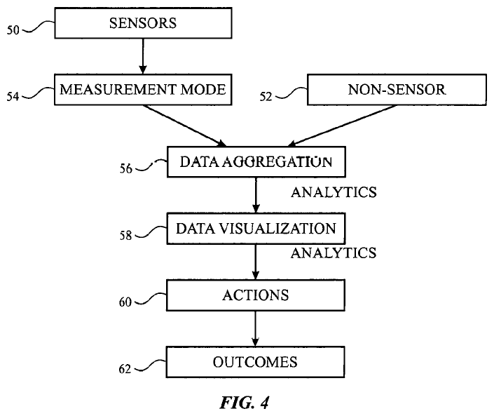

Referring now to FIG. 4, an additional flow chart is provided, to illustrate a method for processing sensor data 50 and non-sensor data 52, according to one embodiment. A measurement mode 54 may be selected for one or more sensors being used. The sensor data 50 and non-sensor data 52 may be aggregated 56 into a relational database. It can be organized in different ways, such as linked to sensor and linked to patient. Each piece of data has a time stamp, but the sampling rate may be different—for example, pollen count may be obtained once a day, but physiologic data every second. Another type of aggregation would be location-based, which could allowed for geographic or population level analysis that may guide health policy or health interventions. For example, regional assessments may be provided based on population clusters and/or various environmental events (e.g. wildfires, storms, drought). The aggregated data 56 may be analyzed to determine or assess relationships between the sensor data and the non-sensor data, and the data may then be displayed, so that the provider/user/patient can visualize the data 58. The data can be shown on a line graph with heart rate and respiratory rates shown over time for nightly data. The median heart rate and respiratory rates for each night can be shown as a way to visual days, weeks, months and/or years' worth of data. Based on the data, a risk assessment may be determined and a status signal or value may be provided. The risk value can also be graphed as a line graph over time separate or with the heart rate and respiratory rate. The risk graph may have shaded regions for the thresholds that will trigger an alert. Non-sensor data can also be shown either on the same graphs to show the variation over time (temperature, pollen count etc.), or shown as a simple red, yellow, green based on the values. Derived parameters such as heart rate multiplied by respiratory rate can also be shown in a graph. Bar charts showing the number of data points received throughout the night can be shows as a quick way to verify the quality of the data and sensor set-up. The data may then be analyzed again to provide one or more recommended actions 60 for the patient (or other user) to follow, which may provide enhanced outcomes 62 for the patient. In one example, on a daily basis, all historical sensor data is correlated with the Air Quality Index (AQI) and based on the r-value of the correlation, the AQI will be factored into the risk value. Each morning when the AQI is received, if the value is greater than 50, it will be used as part of the status or conditional signal, with the coefficient based on the r-value. If the r-value is <0.3 then the coefficient of the AQI in the status signal will be zero, if the r-value is 0.3-0.5 the coefficient will be 2, if the r-value is 0.5-0.7, then coefficient will be 4 and if the r-value is >0.7 the coefficient will be 8. This enables tailoring of the status signal based on the sensitivity of a particular patient to AQI. This also applicable to other air quality or allergen data (retrieved or received from a third party source, such as, but not limited to, the Air Quality Index developed by the Environmental Protection Agency, based on ground-level ozone, particle pollution, carbon monoxide, sulfur dioxide and nitrogen dioxide levels). In addition to adjusting the status signal based on this relationship, the actions may be tailored to let patients, who are known to be sensitive, recommend that they stay indoors when there is poor air quality. The messaging can be adjusted to be even stronger when the air quality if poor and there is deviation from baseline of the patient specific sensor parameters.

The potential non-sensor 52 or sensor 50 systems may be in communication with the processor via wired or wireless communication, including or remote or outdoor data services or sensors and anticipated measurement modes 54 that may be collectively analyzed and presented or visualized to generate particular actions 60 and outcomes 62. Sensors 50 may be used in combination or individually to produce single or multiple measurement modes 54. For example, sound or piezoelectric sensors may be used to measure an individual's cough frequency, breath sound analysis, voice analysis, snoring, sniffle and/or crying. The sensors 50 may be incorporated within the base station 32 or may be external to the base station 32, such as an external sensor placed under and/or attached to the individual or group of individuals, mattress, pillow, bed, house and/or clothing. Each individual sensor will transmit data individually and then all of the data is aggregated together by user, timestamp and potentially geographic location. The data from each sensor can then be visualized individually or can be shown as a combined parameter (such as a composite asthma score combining respiratory rate, heart rate and cough frequency).

In various alternative embodiments, examples of sensors 50 include, but are not limited to, piezocapacitive, piezoresistive, piezometer, barometric, capacitance, current, voltage, resistance or impedance, inductance, elastoresistive, electromagnetic, optical, potentiometric, laser, kinetic inductance, fiber optic, radiofrequency, sonar, opto-acoustic, electro-optical, phototransistor, photodetector, visual light, gyroscopic, stress, strain, bolometer, altimeter, inclinometer, impact, radar, LIDAR, photoelectric, position, rate, calorimetric, ultrasonic, infrared, contact, scintillometric, thermometer, thermistor, pyrometer, image, video, actimeter, accelerometer, gas, spectrometer, opto-chemical, electrochemical, biochemical, olfactory, and time sensors. Measurement modes 54 for the sensors 50 may include, but are not limited to: local atmospheric pressure; regional atmospheric pressure; changes in local atmospheric pressure associated with, but not limited to, geographic location, time, disease status; changes in regional atmospheric pressure associated with, but not limited to, geographic location, time, disease status; differential and changes in temperature between, but not limited to, measurement modes, time, geographic location, disease status; instantaneous and changes in inspiratory and expiratory curves by which effort, work, rate, lung volumes and interactions with cardiac physiology can be determined; frequency, changes, and characterization of cough or sniffle episodes within specified periods of time and interactions with cardiorespiratory physiology; instantaneous and changes in cardiac physiology including estimated venous return, ballistocardiogram, heart rate, function, blood pressure, pulse pressure, and heart rate variability and interactions with respiratory physiology; changes of the relationship between physiologic and pathophysiologic cardiorespiratory conditions associated, but not limited, to geographic location, medication use, time, and disease status; frequency, changes, and characterization of cough episodes within specified periods of time and interactions with cardiorespiratory physiology; relationships between physiologic and pathophysiologic cardiorespiratory conditions; frequency, magnitude, and changes over time of gross body movements; local and regional environmental temperature; global positioning system (GPS) location; exhaled breath temperature; thoracic cavity body temperature; body temperature; local altitude; exhaled breath carbon monoxide, nitric oxide, oxygen, and carbon dioxide; local environmental carbon monoxide, nitric oxide, nitrogen dioxide, oxygen, and carbon dioxide; local and regional air particulate levels; changes in local altitude associated with, but not limited to, geographic location, time, disease status; changes in air particulate levels associated with, but not limited to, geographic location, time, disease status; changes in physical activity levels associated with, but not limited to, cardiorespiratory physiology, geographic location, time, disease status; physical activity levels; satellite images and changes of the regional environment; changes in voice sounds, including, but not limited to pitch, sniffles, and snoring; instantaneous and change in oxygen saturation; sounds in local environment; changes in sounds in local environment; voice sounds; images and change of local environments; changes in sounds emitted during respiratory cycles; sounds emitted during respiratory cycles; local environmental humidity; regional environmental humidity; changes in local environmental humidity; changes in regional environmental humidity; local environmental tobacco smoke; patient body mass and trends; presence or absence of disease-indicative compounds and trends in urine, sputum, blood, or secretions; local and regional levels of disease-aggravating chemicals both airborne and contact; changes in local environmental tobacco smoke; presence or absence of and exposure to dust mites; sleep status, history, and characteristics; levels of or changes in diaphoresis; cardiorespiratory function; changes in cardiorespiratory function; changes in immunologic function or response; and immunologic function or response.

In various alternative embodiments, locations for passive data collection sensors may include bed, mattress, pillow, couch, lamp, bedside table, ceiling, wall, car seat, windshield, doormat, television, television remote control, gaming system, gaming system controls, desktop, desk chair, workstation, computer screen, computer keyboard, computer mouse, tablet computer, mirror, toilet, floor in front of sink, eyeglasses, jewelry, wallet, belt, clothing, watch, watchband, phone, phone screen or interface, toys, toothbrush, steering wheel, purse, handbag, briefcase, shoes, socks, keys, drink mug or cup, silverware, water bottle, headphones, backpack, security camera, security system, body, skin, hearing aid or other ear appliance, oral appliance, contact lenses, medication delivery device, inhaler or the like. These sensor locations may be used individually or in combination. Data captured by the sensors may either be streamed in real-time or recorded at a point in time. If a base station 32 is being used, it may capture and aggregate raw data from these sensors 50 and store the data locally. Full processing, no processing, or limited pre-processing of sensor data can be performed on the base station 32 or sensor system. In the example of audio data, processing may include but is not limited to extracting relevant features from the audio, for example wheezing, changes in pitch, snoring, shortness of breath, sniffling, and/or the frequency content of the relevant sound. The base station 32 or sensor system can display or stream the complete raw data or a subset of the raw-data and the preprocessed features to a remote server.

In various embodiments, any of a number of different devices and method may be used to display data to a patient or other user for visualization 58. For example, data may be displayed via mobile applications, web-based applications, desktop application, visual display on sensor, visual display on base station, lights on sensor, lights on base station, physical gauge on sensor, physical gauge on base station, audible tone from sensor, audible tone from base station, haptic feedback with carried or worn device, haptic feedback on sensor or base station, email message, phone call, fax, video message, video call, audio recording/voicemail, social media, paper mailing, alerts, through or within an electronic health record, or person-to-person meeting.

The method may next provide one or more recommended actions 60 to a patient, healthcare provider, family member and/or other user(s). Such actions may include, but are not limited to, Various actions may be taken to improve disease control, resulting from the measurements obtained from various sensors and the results of algorithmic analysis and/or review and assessment of the system data by patients, caregivers, healthcare providers, and other persons, may include but are not limited to medication regimen adjustment; early initiation of emergent or rescue medications, for example, albuterol and/or oral steroids; avoidance of regional environmental triggers; tobacco cessation; use of allergen-impermeable pillow and mattress covers; washing bedding; removing old carpet; managing home humidity level; washing stuffed animals weekly; removing pets from the home; keeping pets out of bedrooms; properly sealing and storing food; sealing trash containers; regularly cleaning surfaces and floors; regular pest and insect management; assessment of home environment; development and initiation of home remediation plan; education on local (home) and regional environmental triggers; disease self-management education; disease specific education; improving access to medical care; improving coordination of care; change in diet; change in hygiene habits; change in monitoring methods/intensity; adjustment in schedule or routine; scheduling a visit or visits with healthcare provider(s); connecting patient with social network; regulation of local environment temperature; regulation of local environment humidity; regulation of air particulate; and reporting system individual and population level data. Outcomes 62 resulting from the method may include, but are not limited to, reductions in hospitalization; reduction in emergency department visits; reduction in unscheduled office visits; improvement in disease control; improvement in quality of life; reduction in missed school days for children; reduction in missed work days for adults or caregivers; reduction in caregiver stress; improvement in pulmonary function; improvement in medication or treatment plan adherence; reduction in annual reimbursed healthcare expenditures; reduction in rescue inhaler or emergent medication use; improvement in controller medication use; effective use of therapies, including, for example in asthma, oral corticosteroids, inhaled corticosteroids, anti-viral medications; reduction in annual out-of-pocket healthcare expenditures; reduction in activity limitation; reduction in symptom days; reduction in asthma exacerbations; reduction in disability; aid in healthcare provider reporting requirements; and changes in disease-specific policy considerations.

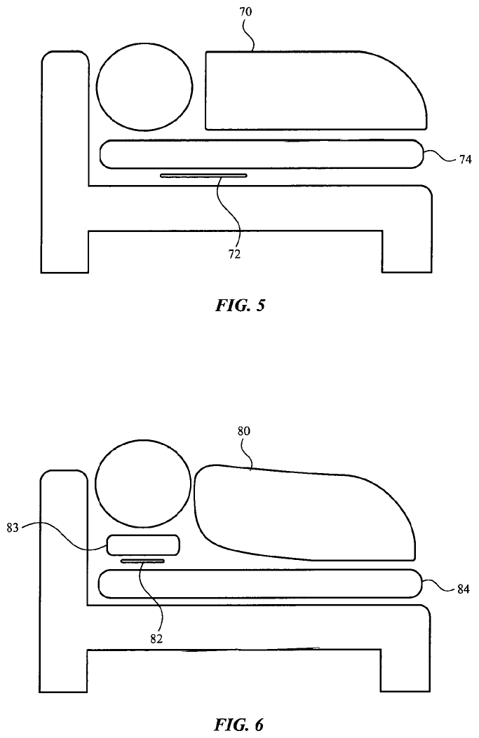

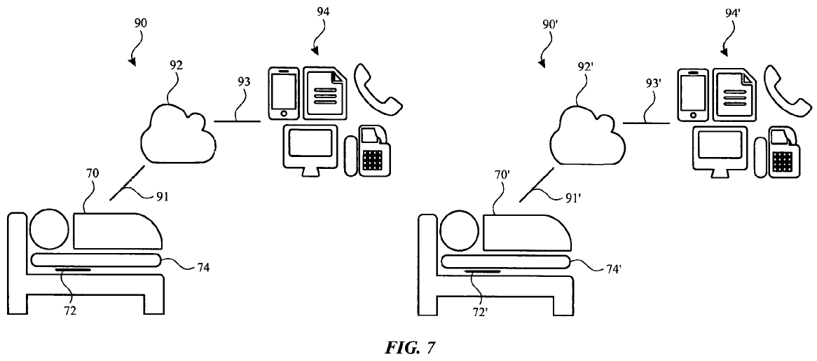

FIGS. 5-7 illustrate an exemplary system for monitoring asthma in a patient. Referring to FIG. 5, in some embodiments, one or more sensors 72 may be placed under a mattress 74 on which the patient 70 sleeps. In an alternative embodiment, shown in FIG. 6, one or more sensors 82 may be placed under a pillow 83 on which a patient 80 sleeps, rather than under a mattress 84. Alternatively or additionally, sensors 72, 82 may be placed under both a mattress and a pillow or elsewhere associated with the bed, in other embodiments. Any suitable combination of sensor placements may be used. FIG. 7 illustrates one embodiment of a monitoring system 90 as a whole. In this embodiment, system 90 includes at least one sensor, which transmits data either to a local base station (not shown) or directly through communications channel 91 to the remote storage and analytics server 92 for processing. The data can then be communicated via communications channel 93 for displayed and visualized via any suitable display device 94. Communications channels 91 and 93 may be hard wired, fiber optic, wireless, and may utilize any available communications. A variety of people, for example data analysts, customer contact personnel, doctors, patients, patient parents or caregivers, payors, medical supply companies and/or others with different roles may be selectively provided with access, analyze or display different aspects of the data or analysis, subject to security and/or controls compliant with privacy and/or protected health information requirements. The analytics server 92 may be an individual or standalone server or computer as described herein, or it may be a cloud service, which has the advantage of providing expandability and access as the number of patients grows. The transmission and storage of health related data, including protected health information will be handled and secured in compliance with the applicable privacy laws and regulatory requirements.

In one embodiment, the analytics server 92 may receive and store the data from multiple patients' sensor, for example 72 and 72′. The raw data from each of the sensors 72 and/or deployed base stations 32 may be stored in one or more files and/or in a database. The remote server may use the sensor data and pre-processed extracted features from each individual or group of individuals and/or external data, including but not limited to, either in combination or separately, weather data, pollen levels, pollution levels, public health records, and other relevant data, to generate a model for each individual or local group of individuals. These models may also be further refined by using aggregate sensor data and pre-processed extracted features collected from all individuals or all groups of individuals. The model may be generated using an algorithm, running on either the server or the base station, which would output data and analysis associated with that individual and their current level of asthma control, including, but not limited to, the frequency of symptoms, the predicted future severity, time period of prediction, and/or confidence level. The algorithm may generate a model for each individual, and that model may be refined on an ongoing basis as the algorithm receives and analyzes more data to more accurately assess the patient's condition. If the individual's present condition and/or predicted future condition passes a pre-defined level (discussed below), the individual, guardian and/or health care provider may be notified either through, for example, text messaging, email, phone call, and/or integration with the electronic medical record. The pre-defined level may be set in a number of ways, including but not limited to, thresholds relative to the patient's baseline statistics, thresholds relative to general population statistics, and/or confidence levels in the personalized model generated by the algorithm.

In some embodiments, the healthcare provider and/or individual or group of individuals can directly provide additional information to the system for use in the algorithm. The individual and/or guardian can record daily symptoms and/or other physiologic measurements, such as peak flow test results, as would normally be recorded in an asthma symptom diary. Healthcare providers can confirm and/or input the individual current asthma condition during or following clinic visits, emergency department visit, and/or hospitalization, to train and validate the model. This feedback will not only improve the accuracy of that particular individual's model, but will also improve all other individuals' models. The machine-learning or other statistical algorithm can also use healthcare data captured from, but not limited to, the individual's electronic medical record, billing records, prescription orders, and/or inhaler or other medication use, to further refine the model.

In some embodiments, the individual and/or group of individuals, guardian and/or the healthcare provider can also monitor the individual's condition through a web page, mobile application, desktop application, and/or accessed through an electronic medical record. Another possible feature is that the system may deliver information, including but not limited to recommendations and/or alerts, to the individual (or the individual's guardian or health care provider) to a mobile or other handheld device. In other embodiments, the system interfaces with existing monitoring equipment at the nursing station, for inpatient settings, including but not limited to the ICU, step-down unit, or other group care environments.

Archiving the data can be performed locally at the base station or sensor, or on the remote server. The system will make the data available to users and/or healthcare providers in pre-defined formats, and the users can request access to the data, specifying, but not limited to, which data, time-range, and/or feature. The remote server may then forward that request to the individual base station 32 or present the data stored on the remote server. In the event that the data is being stored locally, it will be locally processed to satisfy the query, and will return the data to the server, where it then subsequently is displayed to the requestor. This distributed architecture may reduce the storage requirement and may partially reduce the computing resources needed for the remote server, which may subsequently reduce power requirements for system operation.

In some embodiments, the individual data may be configured to differentiate between multiple individuals sleeping in the same bed and for example be able to differentiate between sounds, physiologic parameters, and/or motion from different individuals. Algorithms, including beam forming, may be required to differentiate data sources and produce more reliable, robust, and/or accurate individualized data. Another embodiment is having sensors designed to capture data for each individual.

One example of the above embodiment would be using two or more sensors placed apart from each other—for example at least two sensors that passively detect the heart rate and respiratory rate of individuals who are sleeping on a bed or who are in a room. The following example can also be implemented using sensors placed in various locations acting in a multitude of measurement modes, as described above. Using a machine-learning algorithm that may include, for example, support vector machine and neural networks, the machine can identify the contribution of each individual to the sensor input. This allows the system to have a clean input of the sensor data that is not confounded by other individuals, allowing for easier subsequent processing. This concept may be useful, for example, in identifying the heart rate and respiratory rate of multiple individuals sleeping on the same bed or in the same room. In this example, two more piezoelectric crystals are placed under the bed in opposite corners. The piezoelectric crystals may be mechanically split into quadrants, where the deflection of each quadrant is monitored by sampling its voltage. This allows monitoring for absolute position of the movement in the three dimensions, X-Y-Z. The sampling rate of both piezoelectric crystal sensors is at a sufficient speed such that the delta time difference for a movement to travel different distances to reach both sensors can be captured. When an individual breathes, this causes a deflection of the sensor, which changes in a repeatable pattern in the 3D space. This is also true when the individual heart beats, causing a smaller deflection of the sensor, and also changes in another repeatable pattern in the 3D space. The system would first isolate the aggregate movement of both the movement from the heart beating and also the movement from breathing contributed by each individual as described above. The system would then isolate the movement contributed from the heart beating and the lungs inflating for each individual, using machine learning, including but not limited to support vector machine and neural networks. Once the movement associated with the heart beating and the movement associated with breathing can be extracted for each individual, analysis for individual heart rate and respiratory rate can then be performed. Each of the isolated signals would then be further processed using digital signal techniques and/or artificial learning, and the individual heart rate and respiratory rate can be decoded.

The base station 32 may have accessory features, such as a clock, alarm clock, night-light, and/or music playing capability. This base station 32 could be on a bedside table, attached to the bed, on the wall, ceiling or anywhere else in the room where the individual or group of individuals will be monitored or in any other suitable location, such that the relevant data can be transmitted to and from the base station 32.

There are certain technical challenges in representing and delivering personalized models and monitoring capabilities. The amount of data required to capture and analyze is significant and computationally intensive. A model can be designed for the individual user; however, it would also be beneficial for the system to capture a population level dataset to build a more robust model and provide additional business and clinical value, such as for use in the development of medications and/or population health interventions. The collection and analysis of this dataset may be conducted, for example, through the base station 32 as a node in a distributed computing network. This distributed computing network may provide analytics for other external services not specific to the current intended application, and more specifically, for early detection of asthma exacerbation. Each node may feature local storage and be configured to perform computing. The remote server may act as the master and can pull pre-processed data from the individual nodes to generate a population-based model.

### Methods for Assisting Control of a Medical Condition

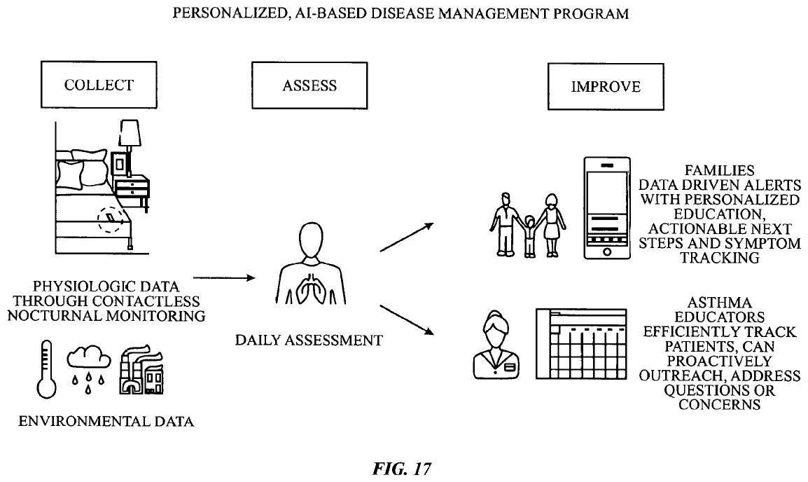

Generally, as described below and summarized in FIG. 17, systems and methods for personalized, artificial intelligence-based disease management can involve collecting physiologic data (e.g., through contactless monitoring, such as nocturnal monitoring), environmental data, and/or other patient information. Such data may be assessed on a regular basis (e.g., daily) and used to provide users such as the patient, a caregiver, medical professionals, asthma educators, etc. with information that can be used to assist control of a patient's medical condition. For example, caregivers (e g , family members) can be provided with data-driven alerts incorporating personalized education, actionable next steps and/or symptom tracking. As another example, asthma educators can be provided with information to track patient status (e.g., identify high risk patients) and/or support the patient by reaching out and addressing questions or concerns regarding the patient and his or her medical condition.

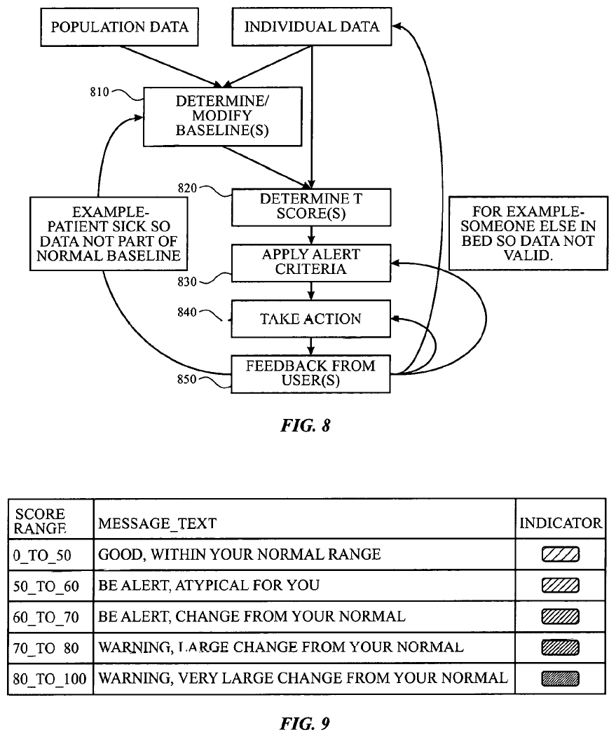

FIG. 8 depicts a flow diagram of an exemplary method for assisting control of a medical condition for a patient. As described in further detail below, population-level data and/or individual patient data may be used to determine and/or modify baseline value(s) for patient parameters (810) such as heart rate, respiratory rate, etc. A “control” signal providing a score value (820) may be generated based at least partially on one or more sensor values and/or baseline value(s). The score value may represent an indication of a user's asthma control, or its opposite or inverse, risk. Significant deviations from an individual's baseline may be translated into increased risk without making an assertion of the certainty, timing, or severity of any worsening in asthma which may occur. As further described herein, the control signal may be calculated utilizing a personalized baseline for each user and may use the engineering concept for a control loop, in which a control response is determined from an “error” signal derived from the contributions of two or more measured and/or derived parameters. The reliability and the predictive ability of the control signal improves the longer the patient is monitored. The control signal may be an analog signal or a digital signal.

The score value may be assessed, such as by applying (e.g., comparing the score value to) one or more alert criteria (830). Based on the application of the alert criteria, certain actions (e.g., generating alert notification messages to a user, prompting user feedback (850) on symptoms, etc.) may be triggered (840). Other aspects of methods for assisting control of a medical condition are described in further detail below.

### Sensor Data

In some embodiments, the system may use a contactless sensor attached to the mattress where someone sleeps to gather data overnight. For example, a contactless sensor is one that does not normally or is not required to contact an individual for making a measurement. The sensor is operationally associated with the patient so that the measurement may be made. This may be physical connection or some other connection such a sound or electromagnetic radiation. The sensor may also be passive, that is not requiring any regular or ongoing overt or conscious user or operator interaction for the sensor to measure a parameter of the user. Of course there may be user interaction to initially set up the sensor, diagnose or fix any exception conditions, and periodically for activities such as battery replacement, cleaning, or operational updates. An example sensor, the Murata SCA11H, is based on the principle of ballistocardiography and reports 10 different parameters: heart rate (FIR), heart rate variability (HRV), beat-to-beat time (B2B), beat-to-beat prime (B2B′), beat-to-beat double prime (B2B″), stroke volume (SV), respiratory rate (RR), signal strength (SS), status (an indication of device function), and time. This sensor also has the option of transmitting the raw signal, which could be further analyzed to detect different types of motion and cough. The sensor operates in a passive mode by being attached to the bed and requiring no user action for measurement to take place. Examples of ways the device can be attached to the bed is attached on the bed frame, under the mattress, on top of the mattress (whether secured by a strap with a pocket or a strip that just lays on the bed), it can be under or over the mattress pad if present, it can either lie directly under where the person sleeps or at the foot or side of the bed. Other sensors may have additional outputs, including sound. Alternative sensors that provide this and/or additional or alternative information may be used, for example radio waves, ultrasonic, or wearable sensors.

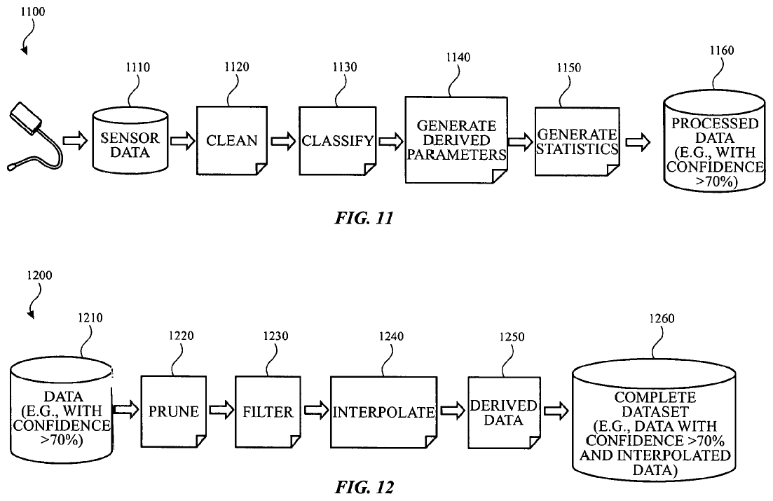

There are several ways that the data can be processed. Raw data received from the sensor may be processed before it is used to calculate the control signal. The processing may include outlier and noise detection/removal and/or data extrapolation to fill in missing data points. One exemplary method 1100 for processing data is shown in FIG. 11, and may be performed periodically to handle gathered data (e.g., daily to handle data gathered from the previous night). Sensor data may be received and/or stored (1110) in one or more suitable computer storage mediums. Such sensor data may be cleaned (1120), such as to identify data that has a sufficient level of confidence. For example, the algorithm may compare the signal strength amplitude of each sample to that of prior samples and assess a difference in the standard deviation among prior samples of signal strength amplitude over a set period of time, or contiguity window. An example cleaning process evaluates over a minimum contiguity window of 10 seconds, evaluating for a predetermined threshold of 2 standard deviations, where full confidence is reached when standard deviation is less than the predetermined threshold of 2 standard deviations for three continuous minutes.

Additionally, the sensor data may be classified (1130). Classification can mean a few things. Generally, it can mean the application of a confidence to a sample to represent how confident one can be that it is a good sample. Confidence of a sample of interest may relate to samples before and after the sample of interest, and how similar the sample of interest is to those other samples. This confidence may be used in later processing steps. Classification can additionally or alternatively mean determining, between two samples that happen to map to the same timestamp, which sample is going to be the “representative sample.” For example, a patient may have two sensors (e.g., patient routinely sleeps in two different beds each with a respective sensor(s), such as a child alternately staying with divorced parents who live separately). In this example, two samples with the same timestamp may be received: a “valid” sample obtained with sensor(s) from the bed in which the patient actually slept, and another sample obtained with sensor(s) from another bed that captured only noise data. In this example, the classification step (1130) classifies which of the two samples is the valid sample. In another example, a user may change a configuration setting such as the time zone for a sensor, after which it is possible that two samples from the same sensor map to the same local time stamp. In this example, the classification step (1130) may decide which of the two samples is the valid. In a third example, two different people (e.g., patient and a guest) may be alternately sleeping in the same bed with sensor(s), and the classification step (1130) may decide which is the valid data sample/data stream. This can be performed, for example, with a simple “default bed” setting that acts as a tie breaker. Additionally or alternatively, the classification step could determine which sample's physiology is most consistent with would be expected for the patient (e.g., with a similarity score).

One or more desired parameters may then be generated or derived from sensor data (1140). For example, parameters may include sleep statistics based on the movement data and other physiologic metrics that are a combination of output parameters like V02 (oxygen consumption) and cardiac output. Additional exemplary derived parameters include contiguity of the data, sums of variables, derivatives, products of variables, the specific shape of a signal (heart rate signal can be seen to have peaks or “humps” during the night, and this shape and height of the humps can be calculated), etc. Statistics on the reported and/or derived parameters may be generated (1150). Exemplary statistics include the mean and/or standard deviation of an individual parameter over a period of time. Example periods are an hour, 12 hour period, 24 hour period, 14 day moving average, though other suitable periods may be used in generating statistics. These parameters can she shown in graph format for easy visualization but can also be depicted in table format that can be easily exported and analyzed further. Furthermore, some or all computed statistics may be stored in a database of processed data (1160). In some variations, only sufficiently accepted data may be stored. For example, data having confidence greater than a predetermined level of confidence (e.g., 70%) can be considered accepted and may be stored in one or more suitable computer storage mediums.

In some variations, as shown in FIG. 12, another exemplary method 1200 may be performed to address issues with sparse or missing data, where reasonable assumptions may be made to interpolate and add to the data set. For example, the method 1200 may process data that has a confidence greater than a specified confidence (for example 70%) and accepted for analysis. Alternatively, the method 1200 may be used on any suitable data (e.g., raw data 1110 described above). As shown in FIG. 12, suitable data may be received and/or stored (1210) in one or more suitable computer storage mediums. The sensor data may be pruned (1220). For example, pruning can be performed to remove “garbage” or undesirable data, such as data having low confidence, and/or drastically unlike neighboring data in such a way that is not physiologically possible. Pruning may, for example, involve comparing a suspect sample to one or more samples with an earlier timestamp and/or with a later timestamp (e.g., removing the suspect sample if it sufficiently deviates from an average sample value for a previous predetermined period of time). For example, if a heart rate sample is 105 beats per minute but average heart rate for the previous minute was about 65 beats per minute, the 105 beats per minute sample may be discarded as bad data. As another example, pruning (1220) may include removing samples when correlated data suggests bad data. For example, if a heartbeat-to-heartbeat time is measured as more than three seconds, this sample may imply a missed heartbeat sample, and thus heart rate and/or respiratory samples obtained at the same time may be determined to be bad data and thus removed.

The sensor data may additionally or alternatively be filtered (1230), such as to remove noisy data (e.g., excessively high or low values are removed, such as via a band pass filter, a high pass filter, a low pass filter, and/or other suitable combination of filters. Missing data may be interpolated (1240), such as with a suitable curve fitting interpolation (e.g., linear polynomial curve-fitting, etc.) to arrive at derived data (1250). The interpolated or derived data, combined with data (1210), can form a sufficiently complete dataset (1260). The complete data set (1260) can further be processed with suitable data processing methods, such as method 1100 described above with reference to FIG. 11.

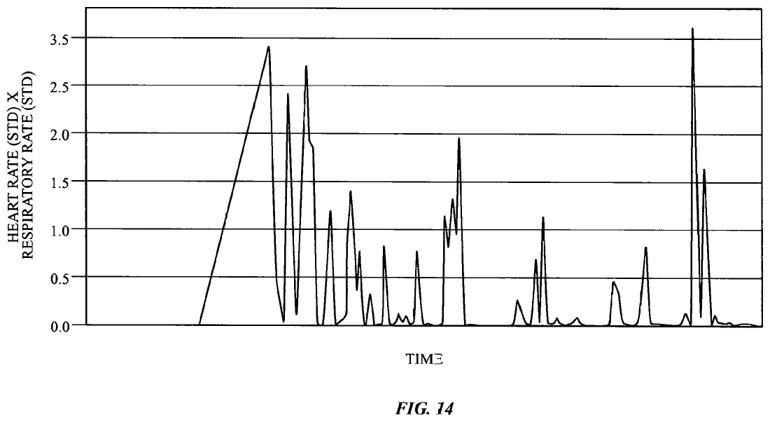

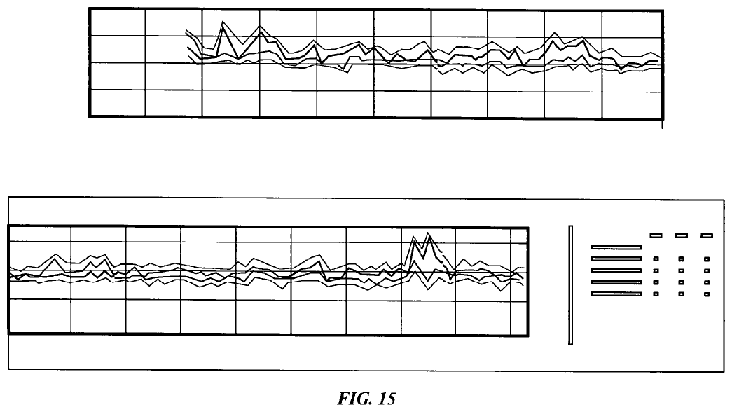

FIG. 14 shows a graph of an exemplary patient's data utilizing multiple derived parameters. Specifically, it shows the product of the standard deviation of heart rate and the standard deviation of the respiratory rate over time. All of the peaks greater than 2.0 correspond with significant asthma symptoms that required albuterol use. The peaks between 1 and 1.5 are related to mild coughs or one episode of vomiting and diarrhea (1/10) which was then followed by a cough. The combination of these two derived parameters allows for higher sensitivity and specificity of significant asthma symptoms. The same patient's data is shown in FIG. 15, expressed as individual heart rate (top) and respiratory rate (bottom) where the shown data is the 50th percentile values for each night). This data (looking individually at heart rate and respiratory rate) is less sensitive and specific than the product of the two derived variables shows in FIG. 14.

Furthermore, derived data can be built using one more many of the incoming data streams to form a new data stream with a different interpretation. The combination of multiple data streams can range from very simple to very complex. In a simple example, a combined stream of heart rate (HR), respiratory rate (RR), and movement data (e.g., from an accelerometer) (and/or other parameters) may be used to create an “IN BED” signal that can be assessed to determine whether the patient is in bed. For example, the HR, RR, and movement data may be combined with the use of a parameterized function (e.g., HR and RR each can have a respective weighting factor, as can a quantified aspect of movement such as magnitude of acceleration). A particular range of values (“in bed” range) of the “IN BED” signal can be correlated with a determination that the patient is in bed. For example, if reasonable HR/RR for this time period has been obtained (e.g., has sufficiently high confidence) and the “IN BED” signal is within the “in bed” range, the patient may be determined to be in bed. As another example, the HR, RR, and movement data (and/or other suitable parameters) can be mapped to an “IN BED” score value as the result of a machine learning model, and the “IN BED” score value can be used to determine whether the patient is in bed. Additionally or alternatively, likelihood that the patient is in bed can utilize previous data (e.g., if the patient was determined in an immediately prior time period to be in bed, the patient is more likely to still be in bed.

Some derived parameters may be based on combinations of other derived parameters. For example, an “ASLEEP” signal may be based on the derived “IN BED” signal or score value described above and the incoming data signal of movement intensity (e.g., accelerometer data). For example, a patient may be determined to be asleep if the “IN BED” signal or score value indicates that the patient is bed for at least a predetermined period of time, and if the movement signal intensity is lower than a predetermined threshold (indicating that the patient is relatively still).

Another example of derived signals is sleep classification (e.g., deep sleep, REM sleep, light sleep, etc.). Yet another exemplary derived signal is a respiratory rate variable, which is not provided by the sensor but may be based on a difference of the current sensor sample from an exponentially weighted average of the signal.

In some variations, derived parameters can be additionally or alternatively based on intra-night data (i.e., sensor data from within a single night of sleep), rather than data across multiple nights. Intra-night data may provide additional or more specific insight that may not be captured when looking at parameters across multiple nights.

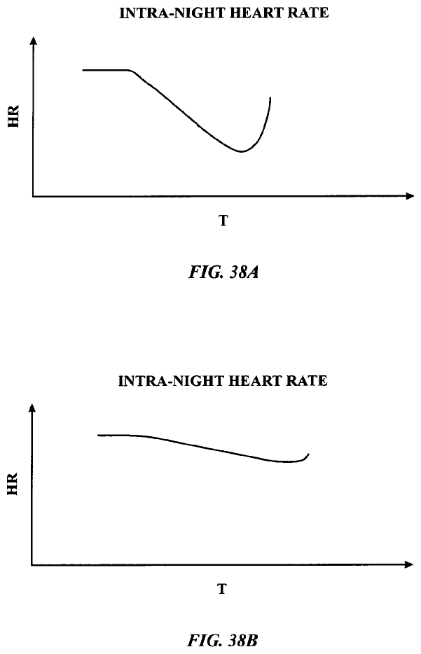

For example, the schematic of FIG. 38A illustrates a typical healthy patient's heart rate pattern over the course of a single night. Typically, heart rate is initially high (e.g., 100 bpm) when the patient is ambulatory. Over the course of a few hours, heart rate gradually slows to a nocturnal rate (e.g., 60 bpm). Shortly before the patient awakes, the heart rises to a value between the initial high value and the nocturnal rate (e.g., 75 bpm). Overall, the healthy patient's heart rate pattern resembles a backwards “J” shape. Within this “J” curve, micro “bumps,” or smaller amplitude deviations within the overall curve, may also be identified. In contrast, the schematic of FIG. 38A illustrates a typical asthmatic patient's heart rate pattern over the course of a single night. Typically, the intra-night heart rate pattern for such a patient has a flatter “J” curve contour that begins high, stays high, and ends high, with only a small dip in the signal. In some instances, micro “bumps” in the “J” curve may also tend to flatten out. Generally, looking at data across multiple nights, a drop in heart rate standard deviation can be a marker of asthma condition if the median heart rate is also high. The intra-night heart rate pattern of FIG. 38B also appears on a macroscale view with a low heart rate standard deviation (due to loss of variability), but the flattening of the “J” curve and/or micro “bumps” in the curve can be characterized and assessed (e.g., in accordance with a trained machine learning model, etc.) as a more specific sign of the asthma condition.

The status signals may be personalized for and/or by each patient and situation. For example, changes may be based on age (heart rate is more responsive when younger), based on genetics (may represent different severity of asthma) or allergen to specific pollen (more significance when that allergen is present).

In some examples, the status signal provides improved signal creation over the use of a single variable. In an alternative aspect, the use of one or more alerting algorithms as described herein may provide additional and improved differentiation of alerts. To understand the breadth of assessment in this invention, when only one assessment criterion is applied to the status signal derived using only the proportional coefficient (kpi) applied to only the shortest term data (Pi), the result is analogous to the simple threshold alarms. However, in some variations the methods described herein may improve upon use of a simple threshold by optionally using a status signal upon multiple time lengths of data, using multiple functions of that data, such as proportional, integral, derivative, and variability (standard deviation) features such as that described herein. Subsequently, one or multiple alert criteria may be applied to such time sequenced status signals.

In some variations, two or more status signals may be computed from one or more non-identical variables. They may then be assessed for alerts or alarms related to different aspects of the patient's disease or for different diseases or conditions which may affect the patient, as described in further detail below.

### Determining Baseline

As shown in FIG. 8, in some variations, a patient's baseline may be determined from population data and/or individual data (810). For example, a patient's baseline may be determined from the combination of expectations based on population-level health data, which may be scaled or normalized for the patient. Such scaling may take into consideration body habitus and/or disease condition, and/or the observations from that individual over a period of time. In one variation, only heart rate is used to establish a baseline. In other variations, other parameters may additionally and/or alternatively be used to establish a baseline.

Population-level health data may, for example, be organized and/or stored in look-up tables including heart rate data (e.g., resting heart rate) for various demographics, such as based on age, gender, weight, BMI, etc. In some variations, individual data contributing to baseline may be based on two (or more) weeks of observation in which the lowest observed HR is assumed to be an indication of baseline. Other amounts of observation time (e.g., three days, five days, one week, etc.) may be a suitable amount of time for providing an indication of baseline. The baseline can be updated over time and, depending on the disease state (such as COPD which has a progressive decline) can be increased or decreased as appropriate.

In some further embodiments, there is an initial training or calibration phase of operation, which may, for example, be a week, two weeks, or other suitable period of time. During this initial phase, no assessment may be made, but the patient may be monitored so that an individual baseline may be established. Alternatively, assessments may be made during the initial phase by comparing to population data and the very limited patient data collected up to that point, but it may only be used to prompt the collection of other data, for example input entered by a user relating to patient symptoms, etc. In further aspects, during the calibration phase, assessments may be performed and compared to population and/or limited patient data, but with a higher criteria or threshold so as to account for lower confidence.

Even beyond the initial learning or calibration phase of operation, it may be useful to continue to include population data in the patient's baseline or to include a comparison to a population baseline data because a patient could be consistently not well controlled and this would be difficult to assess if the only comparison is to the patient's own previous (uncontrolled) state data.

Conventional monitors may define a baseline and use simple threshold cut-off. Even using many different parameters, each with their own cut-off, there is a trade off of sensitivity or specificity—either too many false alarms or missing early changes. In one example as shown in Table 1A, 891 nights' data were analyzed including 161 (18.1%) with asthma symptoms. A random forest (RF) algorithm was applied to sensor data in order to assess the association between the sensor data and the asthma symptoms. The optimal parameter of the RF model was 70 variables at each node. The random forest included 2000 trees. The performance of the RF was based on the Leave One Out validation method, and shows significant improvement over the simple threshold cut-off for heart rate. Using this process, a conventional monitoring system resulted in a sensitivity of about 47.2% (calculated as 76/161), a specificity of about 96.3% (calculated as 703/730), and an accuracy of about 87.4% (calculated as (703+76)/891).

In contrast, using alert thresholds (as further described below, such as in relation to a risk score value) in accordance with the methods and systems described herein provides a much higher sensitivity than that obtained using a random forest model as described above. For example, data shown in Table 1B was generated using methods and systems described herein. Using these processes resulted in a sensitivity of about 85.5% (calculated as 177/207), a specificity of about 50.6% (calculated as 84/166), and an accuracy of about 70% (calculated as (84+177)/373). Sensitivity of the alerts was higher than that achieved with previous random forest models. Additionally, although specificity was lower than that achieved with previous random forest models, this may be considered acceptable because user input and other feedback can be utilized as part of the assessment.

TABLE 1A
performance of RF on nightly data in detecting asthma symptoms

| | Model symptom prediction | | | |
|---|---|---|---|---|
| | | no | yes | TOTAL |
| Symptom | no | 703 | 27 | 730 |
| | yes | 85 | 76 | 161 |
| | TOTAL | 788 | 103 | 891 |

TABLE 2B
performance of risk score value in detecting asthma symptoms

| | Alert | | | |
|---|---|---|---|---|
| | | no | yes | TOTAL |
| Confirmed | no | 84 | 82 | 166 |
| health change | | | | |
| | yes | 30 | 177 | 207 |
| | TOTAL | 114 | 259 | 373 |

### Generating Score Value

As shown in FIG. 8, a score value may be determined (820). For example, in some variations, the system may be configured with a scoring algorithm that utilizes a proportional-integral-derivative (PID) controller, which is traditionally used in closed-feedback control system to produce a correction, which is used to maintain the system at the set point. In these variations, the PID control signal is generated and used as an indicator or process variable to determine the degree of control of a respiratory state or condition. The controller computes a or correction signal or value, which would be the correction that would be applied to the system in a normal PID servo loop. Here, however, the signal may be characterized as a status or condition signal that provides a risk score value that serves:
as a risk indicator. The risk is proportional to the “distance” from the baseline; for example, something which is correlated with risk of exacerbation; and/or
as a response necessity indicator. The larger the control response the more important it is that the user takes it seriously and responds quickly and decisively.

The classic PID controller includes three components assessing the current system status in comparison to the set point:
P=proportional, meaning the difference between the current “position” of the system and the desired position or set-point. In this case it is the difference between the nightly statistics and the baseline statistics.
I=integral, meaning a summation, or the accumulated proportional element over a certain period of time
D=derivative, meaning the slope or rate of change, usually of the proportional element over a certain period of time

Other components could be optionally added; for example, variability (V), meaning the variability of the system variable or state over a period of days. Additional optional control algorithms are known to those skilled in the PID controller art. However, in the systems and methods described herein, the computed correction is not applied directly to the system, but instead used as an indication of risk or control status of the patient. This information may be provided to the medical staff, the parents or other caretakers, and/or the patients themselves.

To calculate the control response, each of the P, I, and D elements are assigned a coefficient determined through a “tuning” process in which the behavior of the controller is judged against the desired performance, the basic form would be:

T_{S}=K_{P}(P)+K_{I}(I)+K_{D}(D)

To improve the performance of the control signal, the P, I, and D elements may be controlled using a composite of days and time periods, each with their own coefficients.

In one embodiment the composite elements are normalized to standard deviations from baseline as follows:

P_{composite}=kₚ₁(P₁)+kₚ₅(P₅)+kₚ₁₀(P₁₀)
P₁ is the simple difference of HR_{baseline}−HR
P₅ is the mean of (HR_{baseline}−HR) over the last 5 days
P₁₀ is the mean of (HR_{baseline}−HR) over the last 10 days

I_{composite}=kᵢ₁(I₁)+kᵢ₅(I₅)+kᵢ₁₀(I₁₀)
I₁ is the sum of (HR_{baseline}−HR) over the last 1 day
I₅ is the sum of (HR_{baseline}−HR) over the last 5 days
I₁₀ is the sum of (HR_{baseline}−HR) over the last 10 days

D_{composite}=k_{d1}(D₁)+k_{d5}(D₅)+k_{d10}(D₁₀)
D₁ is the least squares fit of a line between HR₀ (today) and HR₋₁ (one day prior)
D₅ is the least squares fit of a line between HR₀ (today) and HR₋₅ (five days prior)
D₁₀ is the least squares fit of a line between HR₀ (today) and HR₋₁₀ (ten days prior)

Accordingly, the status or condition signal Ts may be expressed as:

T_{S}=K_{P}(P_{composite})+K_{I}(I_{composite})+K_{D}(D_{composite})+K_{V}(V_{composite})

This signal may be calculated for any variable from any source, including but not exclusively listed in Table 2 below. The status or condition signal T_{S} may be individually tracked. Multiple status signals can be summed between multiple different variables.

The status signal may be computed daily, but could be done over shorter periods of times including every hour or continuously over a trailing time period of 5 minutes, 10 minutes, 15 minutes, 30 minutes 45 minutes, 60 minutes, 90 minutes, 120 minutes, 3 hours, 6 hours 12 hours, 18 hours, 24 hours, 2 days, 3 days, 5 days, 1 week, or more. Multiple scores involving different time frames may be provided to provide information regarding short-term and long-term respiratory status.

In some variations, subjective responses from the patient (or caregiver or other third party), such as regarding the patient's condition (e.g., symptoms), can be used in an automated fashion to adjust the signal. For example, “check-in” messages or alerts to the patient (or caregiver, etc.) may be generated to prompt entry of patient updates. Such messages may be event-driven (e.g., when sensor values sufficiently deviating from baseline values have been measured) and/or periodically or intermittently generated, even when there are no changes from baseline, to see if the patient is experiencing symptoms. For example, if there has been less than 2 standard deviations in the status signal for 4 weeks, a check-in message or alert may be sent. This check-in can be automated through the mobile application such as with a message notification, can be a phone call/text/e-mail from an asthma educator or scheduling of a call with an asthma educator, etc. If the patient (or caregiver) reports that the patient is symptomatic, despite being what was thought to be their baseline, then the baseline is adjusted and variables weighted differently to account for the asthma symptoms. For example, the new baseline could be automatically defined at 0.9× current baseline.

As another example, if the patient/caregiver report that something other than asthma is going on, such as a stomach virus or infection (gastroenteritis), the data may be removed from calculations of the T values, and can also then compare that data to periods where they are symptomatic with asthma to differentiate and automatically eliminate false positives in the future if there are similar characteristics noted with these defined events.

As another example, when a patient/caregiver reports symptoms, those symptoms can be utilized to help automatically tune the thresholds for alerts by weighing variables differently for each individual and also change messaging for different individuals since some patients are very perceptive to symptoms and others that are under-perceivers.

As another example, when a specific trigger is reported and is related to symptoms, in the future when that trigger is reported the analytics can automatically adjust the different weights for parameters after the trigger exposure, such as weighing the D element higher to be more sensitive to any changes that may have occurred as a result of the exposure.

TABLE 2
Exemplary sources and variables for individual status signals

| Source | Variable |
|---|---|
| | Passive bed sensor Heart rate |
| | Respiratory rate |
| | Inspiratory/expiratory ratio or times |
| | Heart rate variability |
| | Relative stroke volume |
| | Movement |
| | Sleep parameters (sleep latency, number of |
| | awakenings, details about sleep cycles) |
| Microphone | Sound (decibel) |
| | Cough |
| | Snoring |
| | Breathing sounds (wheezing or other) |
| Wearable device | Heart rate |
| | Movement (step count) |
| | GPS location |
| Environmental data | Temperature |
| | Humidity |
| | Allergen levels |
| | Pollution levels |
| In-home data (Nest, | Temperature |
| Home Kit etc.) | Humidity |
| Self-reported data | Symptoms |
| | Viral illness |
| | Trigger exposure |
| | Education level (caregiver and/or patient) |
| | Address |
| | Income |
| | Insurance status and type |
| | Medications |
| Population level data | Outbreaks of viral illness (influenza, |
| | rhinovirus etc.) |
| | ER utilization by location |
| | Data from other users within our system in |
| | the same geographical area |
| Medication trackers | Frequency of doses |
| | Locations where doses are administered |
| Home peak flow | |
| or spirometry | |
| Pharmacy data | Medications prescribed |
| | Number and dates of refills |
| Source | Variable |
| Medical claims | ER visits with ICD-10 for asthma |
| | exacerbation |
| | Hospitalizations with ICD-10 for |
| | asthma exacerbation |
| | Clinic visit with ICD-10 for asthma |
| | exacerbation |
| Routine clinic visit | |
| Medical results | Spirometry values |
| | Chest xray read |
| | Allergy testing—blood work or skin test |

In contrast, existing system often utilize simple threshold alert levels, that may result in too high a frequency of false alarms or very high thresholds that may miss the early warning signs.

### Alerts and Actions

As shown in FIG. 8, the score value (e.g., generated with signal Ts described above) can be assessed in accordance with one or more alert criteria (830). In some variations, deviation from baseline (increasing signal) can be correlated with increased likelihood of symptoms and exacerbation. For example, a signal greater than 2 standard deviations may trigger an alert. As another example, a signal greater than 3 standard deviations may trigger a more serious, “major” alert.

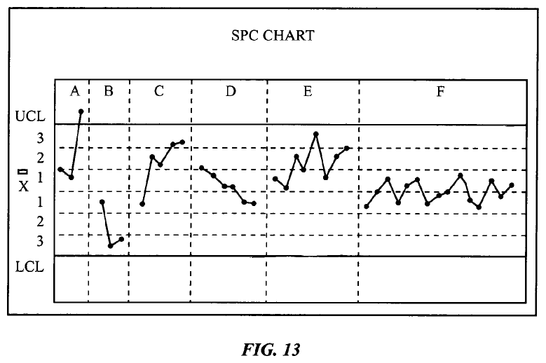

An alternative algorithm to assess the presence of an alert may use process control tools that improve product quality by reducing process variation. Example of detected or out of control conditions may include:
1. If one or more points falls outside of the upper control limit (UCL), or lower control limit (LCL). The UCL and LCL are three standard deviations on either side of the mean—see section A of FIG. 13.
2. If two out of three successive points fall in the area that is beyond two standard deviations from the mean, either above or below—see section B of FIG. 13.
3. If four out of five successive points fall in the area that is beyond one standard deviation from the mean, either above or below—see section C of FIG. 13.
4. If there is a run of six or more points that are all either successively higher or successively lower—see section D of FIG. 13.
5. If eight or more points fall on either side of the mean (some organizations use 7 points, some 9)—see section E of FIG. 13.
6. If 15 points in a row fall within the area on either side of the mean that is one standard deviation from the mean—see section F of FIG. 13.

One example of the upper and lower control limits are the +/−3 standard deviation values.

The definition of alert levels and the application of statistical process control (or an alternative control assessment technique) can be used for a linear or nonlinear mathematical composite, a logical combination, or a neural network combination of the individual parameters that is the sum of the coefficients of the status signals. Analysis based on combinations of multiple parameters may be more informative or reliable than analysis based on individual parameters. For example, asthma is more likely to be present when there are concurrent changes in both respiratory rate and heart rate, than when there is change in only one of these parameters. Alternatively, statistical process control can be applied to individual parameter of the status signals or a multi-day series of individual parameter values to then determine the alerts.

When an alert is triggered it may go directly to the patient (or caregiver if a child), directly to the medical team (including a physician) or a third party service.

When an alert is triggered, a message may be generated to indicate a change in condition or status has been detected. For example, an alert can give details about the change noted and what the change may represent in terms of the disease state. In some variations, the alert may provide education and guidance, and/or direct a change in medications. For example, the alert may direct an individual to look at a medical plan (such as an asthma action plan). As another example, the alert may direct an individual to perform a test, such as a measurement of expiratory flow (e.g., with a peak flow meter) or measurement of oxygen level (e.g., with a pulse oximeter) to give additional clinically relevant information. As yet another example, the alert may instruct the user to be more vigilant in watching for additional symptoms or clinical changes.

The alerts can be a generic alert or can have different levels. In one embodiment the alerts involve categorized alert (e.g., a minor or major alert). In another embodiment as shown in FIG. 9, the alerts may involve a numerically scaled signal from 0 to 100 or some other numerical range or other categorical or range indicia. For example, the signal or alert could also have a corresponding color (e.g. green, yellow and red categories) associated with a risk level.

There can also be alerts that are unrelated to the parameters' values, but instead based on whether data is being obtained. For example, if no data is being received, the patient or user can be notified that no data is being received, and there can be instructions to troubleshoot this problem or seek support (online, phone or other). As another example, an alert may be triggered if there is insufficient data being received, such as less than 1 hour of quality data during a night. This may be an important step, for example, if there is a reliance on this data to be used as part of chronic disease management.

### User Feedback

In some variations, before giving additional information, additional context may be gained through a series of questions, such as asking if there are changes noted in the individual (disease related such as symptoms noted or non-disease related such as a gastroenteritis), asking about medication adherence, potential triggers etc. Information from the patient such as any non-disease related conditions that may have caused an alert can be used to tag that data and have it not used for the analysis of disease state assessment.

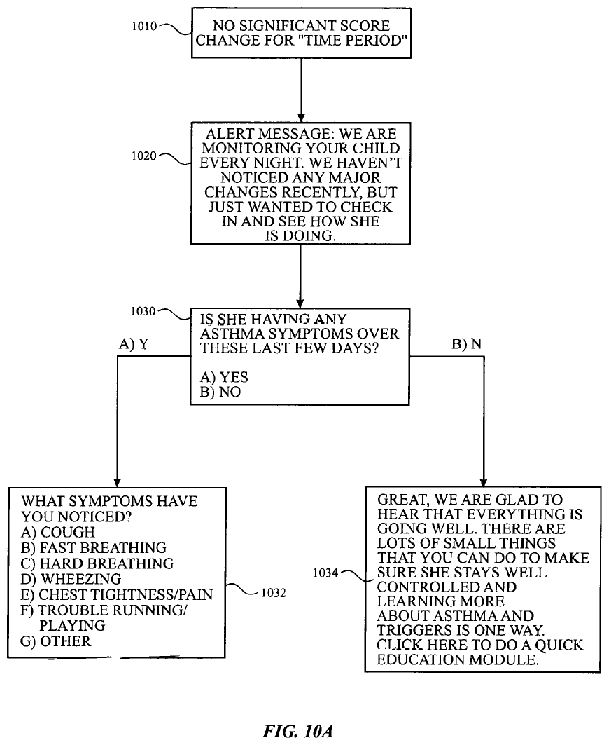

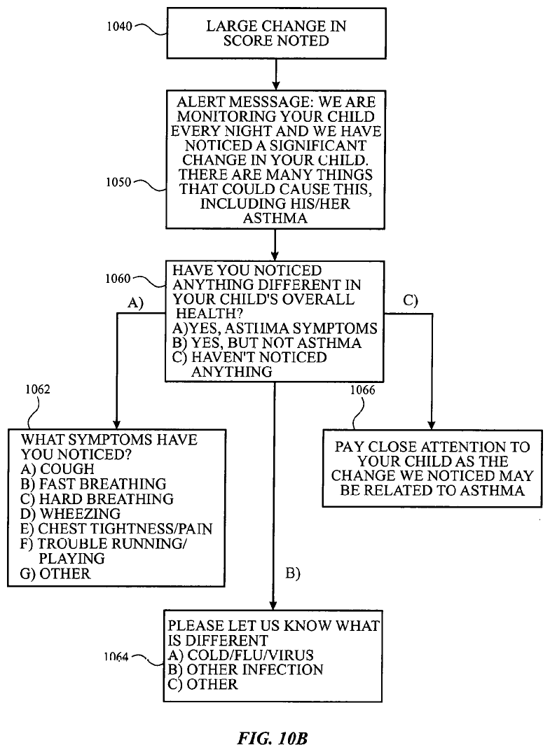

FIGS. 10A and 10B illustrate exemplary workflows of automated interactions with the user based on the responses given to specific questions, in response to alerts.

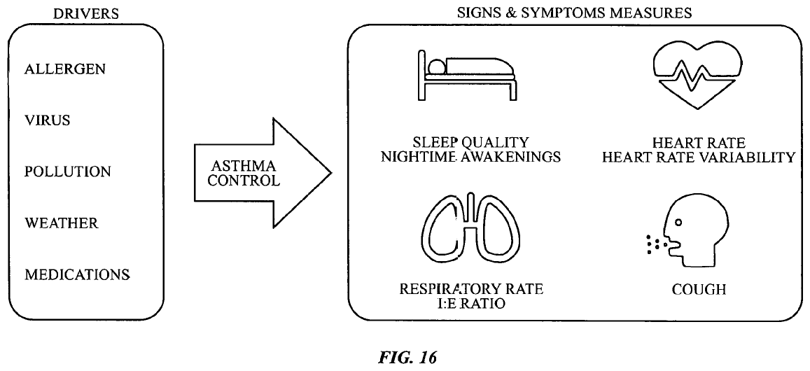

Specifically, FIG. 10A illustrates an exemplary flow of questioning following an alert triggered when there is no significant score change for a predetermined time period (1010). An alert message (1020) may be displayed (e.g., as a message notification in a mobile application, via text message or email, etc.) indicating that no significant changes have been detected, and requesting a patient status update through a series of one or more questions prompting information from a user (e.g., patient or caregiver). For example, the user may be asked an initial question (1030) whether the patient has experienced asthma symptoms recently (e.g., over the previous n days). In response to a user-provided answer confirming that the patient has recently experienced asthma symptoms, the user may be asked a first follow-up question (1032) requesting details regarding type of symptoms experienced. Additional follow-up questions may request additional details regarding frequency, severity, triggers for symptoms, and/or other related aspects. Alternatively, in response to a user-provided answer confirming that the patient has not recently experienced asthma symptoms, the user may be provided with encouragement and/or access to educational information relating to how to maintain control of asthma symptoms (1034). For example, FIG. 16 summarizes exemplary triggers (e.g., allergens, virus, pollution, weather, medication, etc.) that may trigger asthma symptoms and/or other kinds of symptoms. Such triggers may have an effect on sleep quality, derived parameters (e.g., heart rate, heart rate variability, respiratory rate, inspiratory/expiratory ratio) and symptoms (e.g., coughing). Educational modules may be presented to the user to inform the user of such relationships between triggers and parameters and/or symptoms.

FIG. 10B illustrates an exemplary flow of questioning following an alert triggered when a significant change in score has been identified (1040). An alert message (1050) may indicate that a significant change has been detected and may offer a series of questions to investigate any further actions that may be needed to help control patient symptoms. For example, the user may be asked an initial question (1060) whether the user has noticed anything different in the patient's overall recent health. In response to a user-provided answer confirming that the patient has been experiencing asthma symptoms, the user may be asked a first follow-up question (1062) requesting details regarding type of symptoms experienced. Additional follow-up questions may request additional details regarding frequency, severity, triggers for symptoms, and/or other related aspects. Alternatively, in response to a user-provided answer confirming that the patient has been experiencing health changes including symptoms unrelated to asthma, the user may be asked a second follow-up question (1064) requesting additional details regarding the other kinds of symptoms experienced by the patient (e.g., whether the patient has had the cold or flu, other infection, or other kinds of symptoms). Alternatively in response to a user-provided answer confirming that the user has not noticed any health changes, a third follow-up alert (1066) may caution the user to monitor the patient (e.g., as the score change may nevertheless be related to asthma).

### Exemplary Method Utilizing I/E Ratio

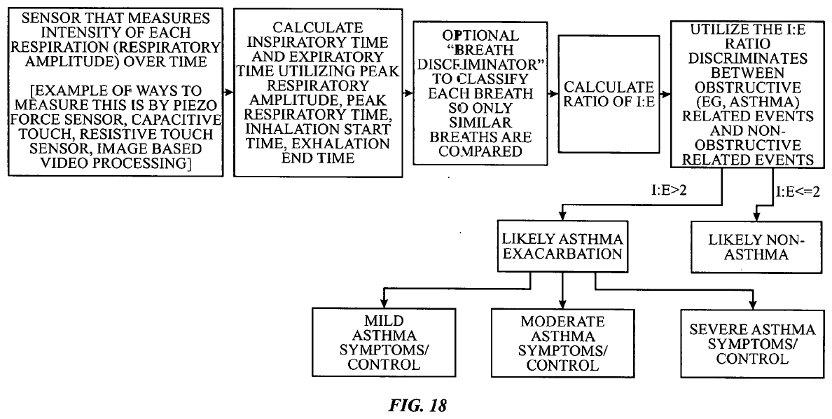

In some variations, as shown in FIG. 18, the monitoring system may be further configured such that the sensor is used to detect inspiratory and expiratory duration, and the system is further configured to determine an inspiratory/expiratory (I/E) ratio for each breath. The I/E ratio can be used to improve discrimination of asthma related events from non-asthma events and/or to improve discrimination of the severity of an asthma events. The detection of inspiratory duration, expiratory duration and/or I/E ratio may be performed in parallel, in sequence or as part of the measurement and assessment of an overall control signal as described above.

These parameters can be evaluated over different time intervals such as each individual breath (typically 3-6 seconds), every minute, every hour and/or every night. The I/E ratio can be evaluated as an average, mode or median across such time intervals. The ratio and/or phase durations can then be compared to standard reference values and/or compared to values from the same individual on prior nights. The comparison may be over the same kinds of reference period (e.g., comparing the median of one night to the median of another night or to the median over a number of nights or sleep periods). For example, the system may perform comparisons for different time ranges, such as comparing the median of night 10 to the average of the medians from nights 1-9 or a range of prior nights.

The I/E ratio may be used as one of the variables in the model to assess control and optionally trigger alerts described above. The I/E ratio may also be used as an independent variable to identify a potential change in disease state, such as respiratory conditions such as asthma where increased I/E ratios represent obstruction and may therefore independently identify worsening asthma control. Other lung diseases where increasing I/E ratio may signal a worsening condition or diseases that are obstructive in nature include chronic obstructive pulmonary disease (COPD) and chronic bronchitis.

Additionally or alternatively, the I/E ratio may be used is in conjunction with the control status signal described above. If an alert is triggered by the control status signal, the I/E ratio may then be assessed to discriminate whether the change noted is likely due to an obstructive (e.g., asthma) or non-obstructive (e.g., non-respiratory related viral illness) condition. The severity of the obstructive condition (e.g., asthma-related event or loss of control) may also be determined or assessed by the I/E ratio (e.g., the larger the ratio, the more severe the obstruction, and therefore more severe the asthma event). For example, daily respiratory data used to compute the daily average I/E ratio may be used in conjunction with self-reported symptom data to determine a personalized I/E threshold, whereby crossing this threshold may be indicate a likelihood that an individual is moving towards becoming symptomatic. This threshold may, for example, be the mid-point between the mean daily I/E ratio when no symptoms are present and the mean daily I/E ratio when respiratory symptoms are present. However, the threshold may be adjusted to meet various sensitivity/specific requirements. Furthermore, in some variations, if an alert is triggered by another status signal indicating a change in health (e.g., score change), the mean daily I/E ratio for that day may be compared against a predetermined, personalized threshold to predict whether this health change is likely related to a change in respiratory health or a more probabilistic, scaled measurement of the likelihood that the health change is related to a change in respiratory health (e.g., an I/E ratio of at least a certain magnitude may be associated with symptoms in, for example, 86% of the patient's prior reported symptoms).

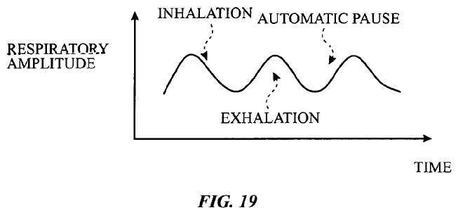

FIG. 19 depicts a normal breathing pattern. The respiratory amplitude can be used a measure of inhalation, based on the change in thoracic volume or respired gaseous volume. When the respiratory amplitude is increasing, this is inspiration. At the end of inspiration there is a peak amplitude, and then a decrease corresponding with expiration. In a normal breathing pattern there is an often a pause at the end of expiration, where there is no expiration or inspiration. Other calculations that may be performed with the respiratory amplitude include, for example, breaths per minute, and ratios of inspiratory to expiratory times. In normal breathing, the expiratory time is typically about equal to or up to about twice as long as the inspiratory time (e.g., I/E ratio ranging between about 1:1 and about 1:2). In a person with airway obstruction, which may be seen in symptomatic asthma, the I/E ratio typically lengthens to about 1:3 or in severe cases as high as 1:5. Accordingly, in some variations, a change toward an extended I/E ratio may generally be correlated in increased likelihood that the change is due to obstructive conditions like asthma.

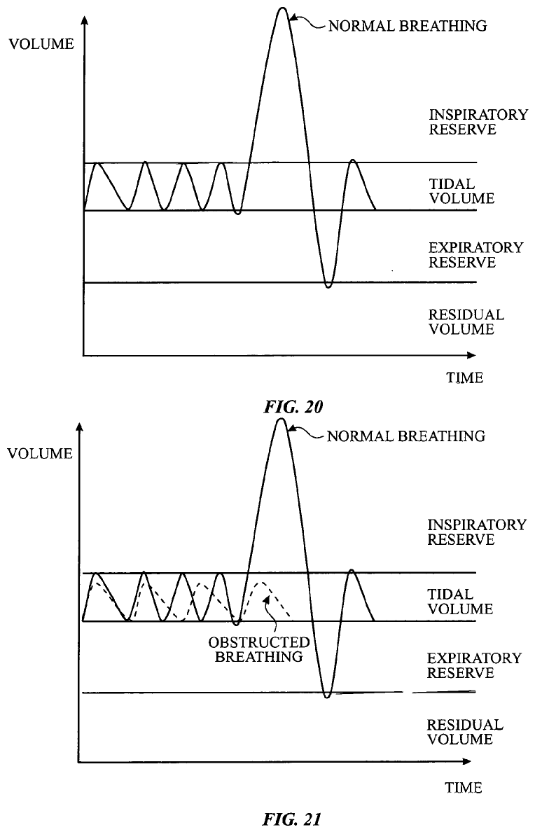

FIG. 20 shows normal breathing reflected in spirometry values achieved when a patient is asked to take a full breath and to fully empty their lungs. It shows the common features measured in spirometry: the vital capacity, the forced vital capacity, and forced expiratory volume. This shows an idealized normal I/E ratio of approximately 1:1. FIG. 21 includes the normal breathing pattern of FIG. 20, overlaid with a spirogram that shows an increased I/E ratio. The thin solid line depicts an idealized I/E ratio (same as FIG. 20). The thick dashed line depicts the conceptual migration toward a prolonged I/E ratio with increased expiration time (when the volume is decreasing), which is seen with airway obstruction. The respiratory amplitude can be used to help determine whether the breath is a typical breath utilizing normal tidal volume or if there was a different pattern such as a deeper breath. In some variations, the I/E ratio can be assessed only on the breaths utilizing the normal tidal volume, such as by using a breath discriminator prior to the assessment. For example, taking breaths with the median or mode respiratory amplitude is likely a way to identify the breaths with a normal tidal volume. Under the typical clinical definition of expiratory time, expiratory time is equal to the expiratory flow time plus the expiratory pause time. However, in some variations, it may be useful to also evaluate the length of the expiratory pause and the length of expiration separately.

There are different ways to determine or calculate the respiratory rate from sensor outputs. The variation in beat-to-beat time of the heart rate may be calculated. The changes in the beat to beat times may be a result of respiration via a phenomenon known as sinus arrhythmia. For example, during inspiration, the thoracic volume is increased, which causes an increase in preload into the heart and subsequent increase in stroke volume. This increase in stroke volume causes a minute increase in beat-to-beat time as the heart takes more time to eject this blood from the heart chamber. Similarly, on expiration, a decrease in beat to beat time is seen. An increased intrathoracic pressure reduces preload or heart filling with a subsequent decrease in stroke volume, which reduced the ejection time. Together, these changes can be detected and the start, end, and transition points of respiration can be distinguished.

Another method of determining respiratory rate is by using a capacitive sensor. For this capacitance method, the working principle is based on capacity change impacted by the human body dielectric properties and movement. Sensing element capacity C can be simplified as flat capacitor

$$C = {ɛ}_{r} {ɛ}_{0} \frac{S}{d} ,$$

where S is electrode area and d is distance between them. This distance is changing during respiratory movements while relative permittivity of the body Er also depends on respiration due the change of amount of air in lungs, thus the body capacity is varying with each breath. From this the start time of inspiration (which is also the end of expiration), and the end time of inspiration (which is also the point of maximum respiratory amplitude and the start time of expiration) can be determined. FIG. 22 is an exemplary schematic illustrating how the capacitance changes during respiration. An example showing the physical phenomena of capacitance change with respiration is described in the reference by Kybartas, Darius, Capacitive sensor for respiratory monitoring. Conference “Biomedical Engineering.” November 2015, the disclosure of which is incorporated herein by reference.

Another method is using a piezo force sensor coupled to the chest, where the sensor can measure changes in force, pressure and/or bend. These can change as a result of inspiration (chest filling with air and increasing in girth) and expiration (air leaving the chest and decreasing in girth). As a result, the starting and ending time for each can be calculated and then used to determine the length of time for inspiration and expiration.

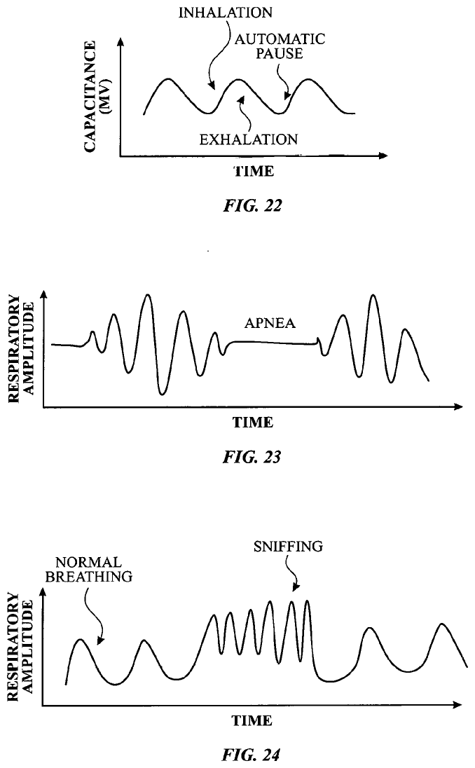

Additionally or alternatively, at least some of the same measurements—respiratory amplitude over time may also be used to identify different types of breathing patterns, such as the Cheyne-Strokes breathing pattern shown in FIG. 23. This breathing pattern may be seen in patients with heart failure, strokes, hyponatremia, traumatic brain injuries and brain tumors. In some instances, it may occur in otherwise healthy people during sleep at high altitudes. It may occur in all forms of toxic metabolic encephalopathy. It is a symptom of carbon monoxide poisoning, along with syncope or coma.

Another example of a respiratory pattern that could be identified is sniffing, this is shown in FIG. 24. The sniffing action results in a sudden change in breathing pattern (shown in the capacitance) due to the change in chest wall movements. In sniffing the frequency increases dramatically for a short period of time and then returns to normal when the sniffing ends and normal respiration resumes. This physical phenomena is discussed in the article by Gonazalez-Sanchez, Carlos et al. Capacitive Sensing for Non-Invasive Breathing and Heart Monitoring in Non-Restrained, Non-Sedated Laboratory Mice. Sensors 2016, 16, 1052, the disclosure of which is incorporated herein by reference.

Table 3 shows example parameters available from two different bed sensors (Murata SCA11H and the BEDDIT® sleep sensor). Based on the additional variables available from the BEDDIT® sleep sensor, utilizing this sensor may allow for improved discrimination of asthma related events (or other chronic disease states). This table also lists multiple different types of sensors and data that may be obtained from them. This is not an exhaustive list, many other types of sensors could be used as part of this system, including specifically for determining the I/E ratio, one example is the Microsoft Kinect motion sensing input devices.

TABLE 3
Example parameters from multiple different sources

| | | | | 3^{rd} Party Data |
|---|---|---|---|---|
| | | | | (via stationary |
| | | | | address or |
| | Murata | | Connected | phone/watch |
| | SCA111 | Beddit | Home | GPS) |
| Physiologic | | | | |
| Heart rate | ✓ | ✓ | | |
| Heart rate variability | ✓ | ✓ | | |
| Respiratory rate | ✓ | ✓ | | |
| Respiratory cycles | | ✓ | | |
| Respiratory cycles Amp | | ✓ | | |
| Sleep measures | | ✓ | | |
| Local/Home | | | | |
| Temperature | | ✓ | ✓ | |
| Humidity | | ✓ | ✓ | |
| Air quality | | | ✓ | |
| Vacuum Cleaner | | | ✓ | |
| Outside environment | | | | |
| Weather | | | | ✓ |
| Pollution | | | | ✓ |
| Allergens | | | | ✓ |

### System Components

Methods described herein may be implemented at least in part by one or more suitable computing devices (e.g., controller and/or processor in communication with a memory device or other computer storage product, various one or more output or input devices, etc.). Examples of such components are described below.

For example, the controller may be implemented with numerous general purpose or special purpose computing systems or configurations. Various exemplary computing systems, environments, and/or configurations that may be suitable for use with the systems and devices disclosed herein may include, but are not limited to software or other components within or embodied on personal computing devices, network appliances, servers, or server computing devices such as routing/connectivity components, portable (e.g., hand-held) or laptop devices, multiprocessor systems, microprocessor-based systems, and distributed computing networks. Examples of portable computing devices include smartphones, personal digital assistants (PDAs), cell phones, tablet PCs, phablets (personal computing devices that are larger than a smartphone, but smaller than a tablet), wearable computers taking the form of smartwatches, portable music devices, and the like, and portable or wearable augmented reality devices that interface with an operator's environment through sensors and may use head-mounted displays for visualization, eye gaze tracking, and user input.

The processor may be any suitable processing device configured to run and/or execute a set of instructions or code and may include one or more data processors, image processors, graphics processing units, physics processing units, digital signal processors, and/or central processing units. The processor may be, for example, a general-purpose processor, Field Programmable Gate Array (FPGA), an Application Specific Integrated Circuit (ASIC), and the like. The processor may be configured to run and/or execute application processes and/or other modules, processes and/or functions associated with the system and/or a network associated therewith. The underlying device technologies may be provided in a variety of component types, e.g., metal-oxide semiconductor field-effect transistor (MOSFET) technologies like complementary metal-oxide semiconductor (CMOS), bipolar technologies like emitter-coupled logic (ECL), polymer technologies (e.g., silicon-conjugated polymer and metal-conjugated polymer-metal structures), mixed analog and digital, and the like.

In some variations, the memory may include a database (not shown) and may be, for example, a random access memory (RAM), a memory buffer, a hard drive, an erasable programmable read-only memory (EPROM), an electrically erasable read-only memory (EEPROM), a read-only memory (ROM), Flash memory, and the like. As used herein, database refers to a data storage resource. The memory may store instructions to cause the processor to execute modules, processes and/or functions associated with the processing device (108), such as ballistocardiogram signal data processing, communication, display, and/or user settings. In some variations, storage may be network-based and accessible for one or more authorized users. Network-based storage may be referred to as remote data storage or cloud data storage. ECG signal data stored in cloud data storage (e.g., database) may be accessible to respective users via a network, such as the Internet. In some variations, database may be a cloud-based FPGA.

Some variations described herein relate to a computer storage product with a non-transitory computer-readable medium (also may be referred to as a non-transitory processor-readable medium) having instructions or computer code thereon for performing various computer-implemented operations. The computer-readable medium (or processor-readable medium) is non-transitory in the sense that it does not include transitory propagating signals per se (e.g., a propagating electromagnetic wave carrying information on a transmission medium such as space or a cable). The media and computer code (also may be referred to as code or algorithm) may be those designed and constructed for a specific purpose or purposes. Examples of non-transitory computer-readable media include, but are not limited to, magnetic storage media such as hard disks; optical storage media; holographic devices; magneto-optical storage media such as optical disks; solid state storage devices such as a solid state drive (SSD) and a solid state hybrid drive (SSHD); carrier wave signal processing modules; and hardware devices that are specially configured to store and execute program code, such as Application-Specific Integrated Circuits (ASICs), Programmable Logic Devices (PLDs), Read-Only Memory (ROM), and Random-Access Memory (RAM) devices. Other variations described herein relate to a computer program product, which may include, for example, the instructions and/or computer code disclosed herein.

The systems, devices, and/or methods described herein may be performed by software (executed on hardware), hardware, or a combination thereof. Hardware modules may include, for example, a general-purpose processor (or microprocessor or microcontroller), a field programmable gate array (FPGA), and/or an application specific integrated circuit (ASIC). Software modules (executed on hardware) may be expressed in a variety of software languages (e.g., computer code), including C, C++, JAVA®, Python, Ruby, VISUAL BASIC®, and/or other object-oriented, procedural, or other programming language and development tools. Examples of computer code include, but are not limited to, micro-code or micro-instructions, machine instructions, such as produced by a compiler, code used to produce a web service, and files containing higher-level instructions that are executed by a computer using an interpreter. Additional examples of computer code include, but are not limited to, control signals, encrypted code, and compressed code.

A user interface may permit an operator to interact with and/or control the processing device directly and/or remotely. For example, the user interface may include an input device for an operator to input commands and an output device for an operator and/or other observers to receive output (e.g., view patient data on a display device) related to operation of the processing device. One or more graphical user interfaces (GUIs), such as those described below, can be displayed for user interaction.

User interface may serve as a communication interface between an operator and the processing device. In some variations, the user interface may comprise an input device and output device (e.g., touch screen and display) and be configured to receive input data and output data from one or more of the ballistocardiogram device, computing devices, input device, and output device. For example, ballistocardiogram signal data generated by ballistocardiogram device may be processed by controller and displayed by the output device (e.g., monitor display). As another example, operator control of an input device (e.g., joystick, keyboard, touch screen) may be received by user interface and then processed by controller for user interface to output a control signal to one or more of the processing device and ECG device.

An output device of a user interface may output ballistocardiogram signal data corresponding to a subject, and may comprise one or more of a display device and audio device. The display device may be configured to display a graphical user interface (GUI). A display device may permit an operator to view ballistocardiogram signal data and/or other data processed by the controller. In some variations, an output device may comprise a display device including one or more of a light emitting diode (LED), liquid crystal display (LCD), electroluminescent display (ELD), plasma display panel (PDP), thin film transistor (TFT), organic light emitting diodes (OLED), electronic paper/e-ink display, laser display, and holographic display.

An audio device may audibly output subject data, sensor data, system data, alarms and/or warnings. In some variations, an audio device may comprise at least one of a speaker, piezoelectric audio device, magnetostrictive speaker, and/or digital speaker. In some variations, an operator may communicate with other users using the audio device and a communication channel For example, the operator may form an audio communication channel (e.g., VoIP call) with a remote operator, ECG technician, and/or subject.

Some variations of an input device may comprise at least one switch configured to generate a control signal. For example, an input device may comprise a touch surface for an operator to provide input (e.g., finger contact to the touch surface) corresponding to a control signal. An input device comprising a touch surface may be configured to detect contact and movement on the touch surface using any of a plurality of touch sensitivity technologies including capacitive, resistive, infrared, optical imaging, dispersive signal, acoustic pulse recognition, and surface acoustic wave technologies. In variations of an input device comprising at least one switch, a switch may comprise, for example, at least one of a button (e.g., hard key, soft key), touch surface, keyboard, analog stick (e.g., joystick), directional pad, pointing device (e.g., mouse), trackball, jog dial, step switch, rocker switch, pointer device (e.g., stylus), motion sensor, image sensor, and microphone. A motion sensor may receive operator movement data from an optical sensor and classify an operator gesture as a control signal. A microphone may receive audio and recognize an operator voice as a control signal. The input device could also be constantly measuring and sending data without a control switch, but can communicate via other devices such as a PC, tablet, phone, nursing station monitor system.

As depicted in FIG. 1, a processing device described herein may communicate with one or more networks and computing devices through a network interface. In some variations, the processing device may be in communication with other devices via one or more wired and/or wireless networks. For example, the network interface may permit the processing device to communicate with one or more of a network (e.g., Internet), remote server, and database. The network interface may facilitate communication with other devices over one or more external ports (e.g., Universal Serial Bus (USB), multi-pin connector) configured to couple directly to other devices or indirectly over a network (e.g., the Internet, wireless LAN).

In some variations, the network interface may comprise radiofrequency (RF) circuitry (e.g., RF transceiver) including one or more of a receiver, transmitter, and/or optical (e.g., infrared) receiver and transmitter configured to communicate with one or more devices and/or networks. RF circuitry may receive and transmit RF signals (e.g., electromagnetic signals). The RF circuitry converts electrical signals to/from electromagnetic signals and communicates with communications networks and other communications devices via the electromagnetic signals. The RF circuitry may include one or more of an antenna system, an RF transceiver, one or more amplifiers, a tuner, one or more oscillators, a digital signal processor, a CODEC chipset, a subscriber identity module (SIM) card, memory, and the like. A wireless network may refer to any type of digital network that is not connected by cables of any kind. Examples of wireless communication in a wireless network include, but are not limited to cellular, radio, satellite, and microwave communication. The wireless communication may use any of a plurality of communications standards, protocols and technologies, including but not limited to Global System for Mobile Communications (GSM), Enhanced Data GSM Environment (EDGE), high-speed downlink packet access (HSDPA), wideband code division multiple access (W-CDMA), code division multiple access (CDMA), time division multiple access (TDMA), Bluetooth, Wireless Fidelity (Wi-Fi) (e.g., IEEE 802.11a, IEEE 802.11b, IEEE 802.11g and/or IEEE 802.11n), voice over Internet Protocol (VoIP), Wi-MAX, a protocol for email (e.g., Internet Message Access Protocol (IMAP) and/or Post Office Protocol (POP)), instant messaging (e.g., eXtensible Messaging and Presence Protocol (XMPP), Session Initiation Protocol for Instant Messaging and Presence Leveraging Extensions (SIMPLE), and/or Instant Messaging and Presence Service (IMPS)), and/or Short Message Service (SMS), or any other suitable communication protocol. Some wireless network deployments combine networks from multiple cellular networks or use a mix of cellular, Wi-Fi, and satellite communication. In some variations, a wireless network may connect to a wired network in order to interface with the Internet, other carrier voice and data networks, business networks, and personal networks. A wired network is typically carried over copper twisted pair, coaxial cable, and/or fiber optic cables. There are many different types of wired networks including wide area networks (WAN), metropolitan area networks (MAN), local area networks (LAN), Internet area networks (IAN), campus area networks (CAN), global area networks (GAN), like the Internet, and virtual private networks (VPN). As used herein, network refers to any combination of wireless, wired, public, and private data networks that are typically interconnected through the Internet, to provide a unified networking and information access system.

### Exemplary GUIs

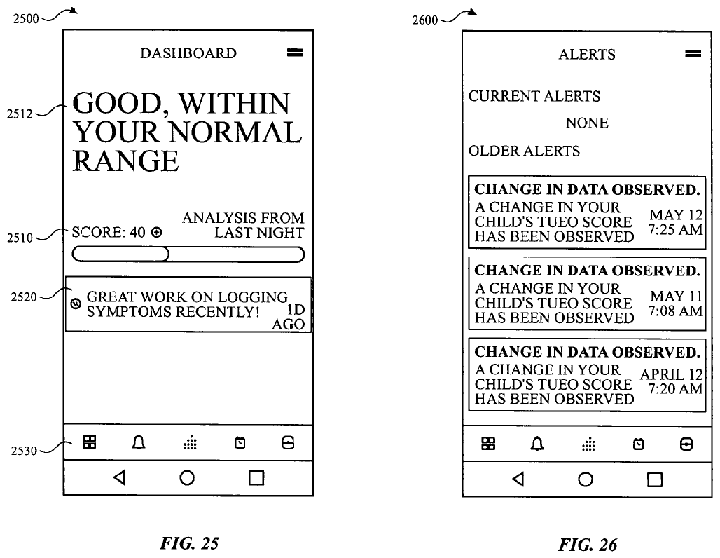

Generally, FIGS. 25 through 34 depict exemplary graphical user interfaces (GUIs) that may be displayed to a user (e.g., on a mobile device via a mobile application) to assist in controlling a medical condition such as asthma.

FIG. 25 is an exemplary GUI 2500 displaying a dashboard highlighting high-level patient status and other information for a user. The GUI 2500 may, for example, be displayed as a primary or “home” screen of a mobile application. The GUI 2500 may display a qualitative assessment 2512 of a risk score value (e.g., calculated as described above) (“Good, within your normal range”) and/or a numerical indication 2510 of the score value. The GUI 2500 may also display one or more alerts 2520 (or other suitable notifications) to direct the user's attention to certain information. Furthermore, the GUI 2500 may display a navigable menu 2530 providing access to one or more GUIs and functionalities, such as an alerts listing, data and/or symptoms tracking, medical condition (e.g., asthma) related tools, and/or educational learning modules.

FIG. 26 is an exemplary GUI 2600 displaying an alerts menu providing a listing of alerts applicable to the patient. The listing may include separate sections for currently applicable alerts and previously-issued alerts. As shown in FIG. 26, one exemplary kind of alert indicates when a significant change (e.g., exceeding a predetermined threshold) in risk score value has been determined. Each alert may be date-stamped and/or time-stamped. In some variations, alerts may be color-coded to indicate severity of the alert. For example, a large change may be communicated in a red-colored alert, while a smaller change may be communicated in a yellow-colored alert. Other visual aspects (e.g., font size) of the alert may additionally or alternatively be adjusted based on severity of the alert.

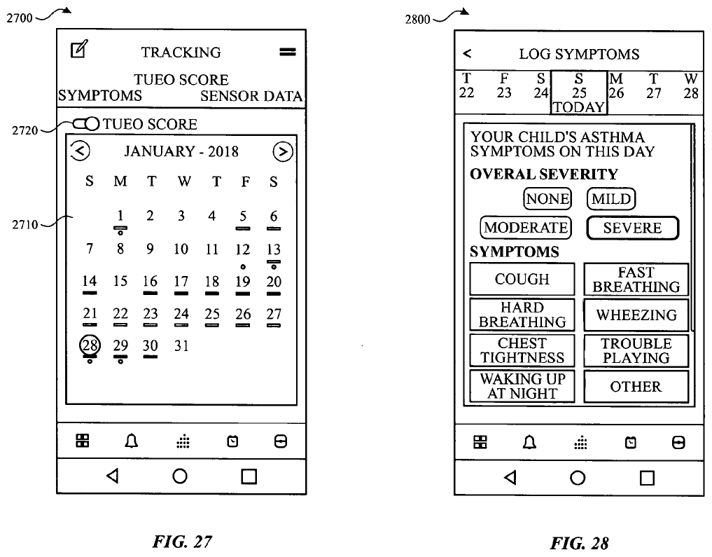

FIG. 27 is an exemplary GUI 2700 displaying a tracking menu (e.g., for tracking data and/or symptoms) including a navigable calendar view. The calendar view 2710 may display summary data and/or symptom information. Individual days in the calendar view 2710 may be selectable to enter and/or edit symptom and/or other date-specific information. As shown in FIG. 27, certain days having associated logged symptom information may be designated with a marker (e.g., a dot). In some variations, specific symptom types as logged may be indicated on certain days (e.g., coughing may be indicated with a graphical representation of a cough, such as a graphical gust of air). Additionally or alternatively, risk score values for at least some of the displayed days may be summarized in the calendar view 2710, such as by displaying a numerical score value and/or by color-coding the calendar date, where the color code may be correlated with severity of risk score value. The appearance of risk score values and/or color coding may be toggled on and off with a selector 2720 or other user interface element.

FIG. 28 is an exemplary GUI 2800 displaying a user interface for logging symptoms (e.g., as another feature of a tracking menu) for a particular day. The GUI 2800 may be accessible, for example, by selecting a date in the calendar view of GUI 2700 shown in FIG. 27. A desired date may additionally or alternatively be accessed by a drop-down menu, scroll wheel, or any suitable user interface element. As shown in FIG. 28, the GUI 2800 may be prepopulated with selectable answers to a predetermined set of questions. For example, a user may be prompted to select a designation for severity of general asthma symptoms (e.g., none, mild, moderate, severe). Additionally or alternatively, a user may be prompted to select one or more types of predetermined categories of symptoms (e.g., cough, fast breathing, hard breathing, wheezing, chest tightness, waking up at night, trouble playing, etc.) experienced by the patient. The predetermined severity designations and categories of symptom types may facilitate standardization and consistency of symptom logging across different days, thereby enabling accurate comparisons over time. Furthermore, GUI 2800 may provide a text box in which the user may enter other text-based notes. Following entry of symptoms for the selected day, the symptom-related data may be stored and subsequently available for reference through a tracking menu such as the calendar view of GUI 2700 shown in FIG. 27.

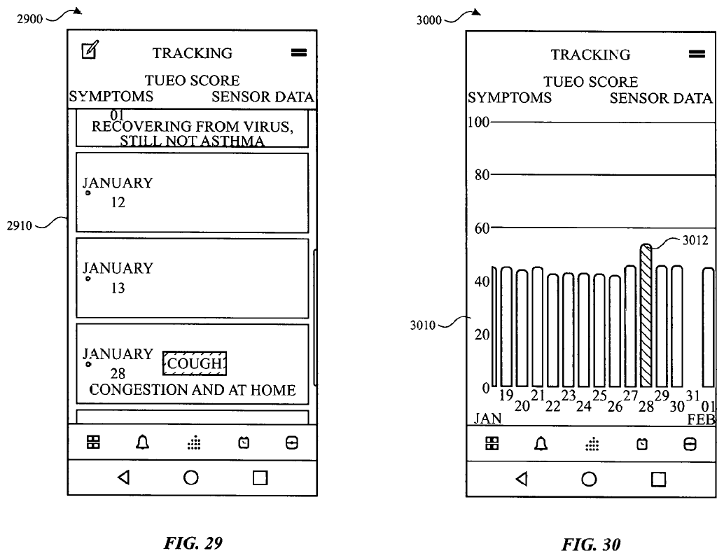

FIG. 29 is an exemplary GUI 2900 displaying a tracking menu including a navigable list view 2910. The list view 2910 may be similar in content to the calendar view 2710 by displaying summary data and/or symptom information, except that the list view 2910 may be spacious enough to display additional details (e.g., text-based description of logged symptoms). Selection of a particular date in the list view 2910 may enable access to a logging GUI such as GUI 2800, similar to selection of a particular day in the calendar view 2710. In some variations, the list view 2910 may be filterable (e.g., selected to display only days on which data such risk score and/or logged symptoms are available for viewing). The symptom list view may display dates and associated information in chronological (or reverse chronological) order.

FIG. 30 is an exemplary GUI 3000 displaying a tracking menu communicating risk score value. GUI 3000 may include, for example, a quantitative bar chart with bars indicating numerical risk score value over time on certain days. The bars may be individually labeled with risk score values (e.g., with a label that is continuous displayed or displayed upon selection or hovering over the bar, etc.) and/or color-coded based on risk score value. For example, bar 3012 is over a certain risk threshold (e.g., 50) and is accordingly colored differently than the other bars in the chart 3010, so as to more clearly distinguish that a higher risk score value was measured on that day compared to others.

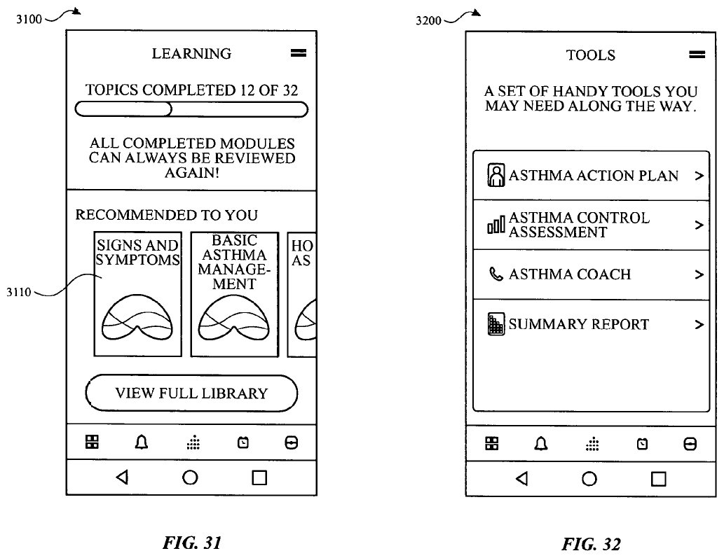

FIG. 31 is an exemplary GUI 3100 displaying a learning modules menu. GUI 3100 may include one or more icons 3110 corresponding to respective educational modules that, when selected, may provide helpful health-related information to a user (e.g., in the form of text, audio, and/or video, etc.). GUI 3100 may display a full library of available modules, and/or a personalized or tailored subset of learning modules based on predicted relevance to a user, such as based on content of symptom logging, elevated risk score values, and/or other information from the user and/or patient. For example, a module relating to how to distinguish between different asthma symptoms may be recommended to a beginning user who may need to become familiarized with symptom-related terminology. As another example, a module may relate to how to reduce environmental triggers. Such modules may be automatically recommended to a user through the learning modules menu, and/or may be suggested during the course of user questioning (e.g., after the user responds “Yes” to a prompted question whether the patient is experiencing asthma symptoms, a module relating to how to reduce environmental triggers for asthma may be suggested to the user). Furthermore, a user's progress in completing the recommended (or full library) of learning modules may be tracked and displayed to the user.

FIG. 32 is an exemplary GUI 3200 displaying a tools menu. The tools menu may include access to one or more features for improving control of the patient's medical condition. For example, GUI 3200 may include a selectable listed item that provides access to a stored asthma action plan outlining medication dosage, and other guidelines from the patient's physician or medical professional. In other examples, GUI 3200 may provide links to an asthma control assessment (e.g., interactive questionnaire), an asthma education or other coach (e.g., provide an avenue for a text message combination or provide other contact info), and/or a summary report providing an overview of patient status, data, and/or symptoms, etc.

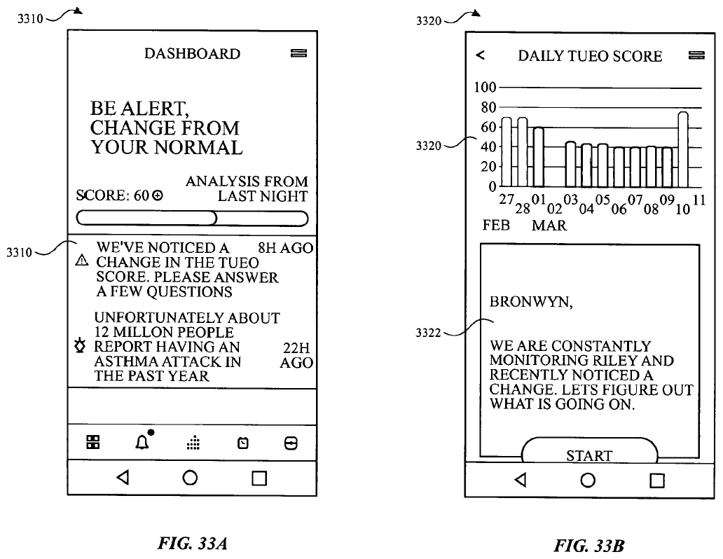

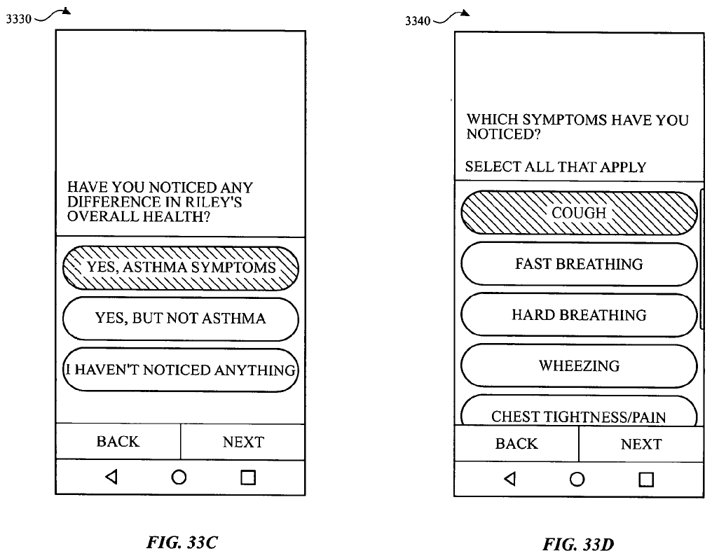

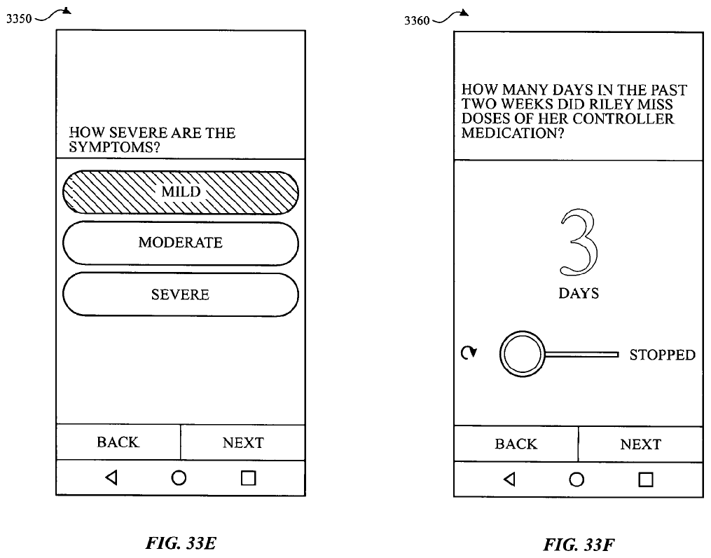

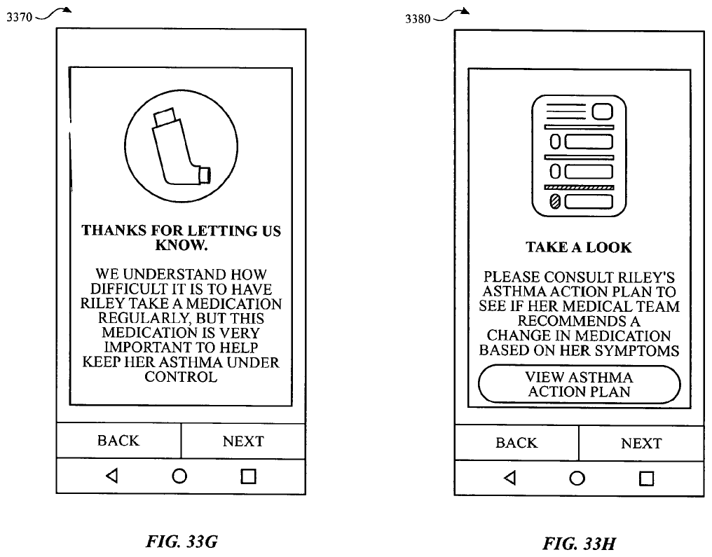

FIGS. 33A-33H are exemplary GUIs as part of an interactive user workflow in which the user is prompted to provide specific health-related information (e.g., symptoms, environmental information, etc.), such as in response to an alert. The interactive user workflow may begin, for example, with GUI 3310 shown in FIG. 33A displaying a dashboard and an alert 3310. In this example, the alert is triggered based on a significant change in risk score value compared to patient baseline. In response to the user selecting the alert 3310, the GUI 3320 shown in FIG. 33B may be displayed.

As shown in FIG. 33B, GUI 3320 includes information related to the selected alert 3310. In this example, GUI 3320 displays a timeline 3320 of risk score values, which visualizes the change in risk score value that triggered the alert 3310. Additionally or alternatively, GUI 3320 may display a prompt 3322 to guide the user through a series of questions in a user interface workflow. The user interface workflow may have a decision tree-like structure navigated by the user selecting from a predetermined set of responses. In this example, the series of questions may be designed to investigate potential reasons for the change in risk score value. An exemplary prompt in the user interface workflow is shown in GUI 3330 in FIG. 33C. Specifically, the example of GUI 3330 prompts the user to answer whether he or she has noticed any difference in the patient's overall health, and presents a number of predetermined, selectable responses (e.g., “Yes, asthma symptoms”, “Yes, but not asthma”, and “I haven't noticed anything”).

If, for example, the user selects “Yes, asthma symptoms” in GUI 3330, the GUI 3340 shown in FIG. 33D may be displayed. In this example, GUI 3340 prompts the user to select any one or more symptoms from a predetermined list of asthma symptoms that the user has noticed in the patient (e.g., “cough”, “fast breathing”, “hard breathing”, “wheezing”, “chest tightness or pain”, etc.). In some variations, GUI 3340 may additionally or alternatively provide a text field for the user to manually enter one or more symptoms. In response to a user selection of one or more asthma symptoms, GUI 3350 shown in FIG. 33E may be displayed. GUI 3350 prompts the user to characterize severity of the selected symptoms (e.g., “Mild”, “Moderate”, “Severe”). Although GUI 3350 prompts the user to characterize the group of selected asthma symptoms collectively as a whole, in other variations the user may be prompted to characterize severity of each individual asthma symptom, such that different selected asthma symptoms may be indicated as having different severities.

FIG. 33F is an exemplary GUI 3360 displaying an additional follow-up question prompting the user to enter information relating to patient compliance with medication. For example, GUI 3360 may prompt the user to enter how many days in the past two weeks (or other period of time) the patient missed asthma controller medication doses. The user may respond by adjusting a slider bar as shown in FIG. 33F, entering a text value, incrementing a scroll wheel or arrow icons, dictation, and/or in any suitable manner.

FIG. 33G is an exemplary GUI 3370 displayed in response to the user-entered information in the user interface workflow relating to an alert. In this example, following confirmation by the user that the patient has recently missed medication doses and experience asthma symptoms, GUI 3370 may display a reminder of the importance of adherence to a medication plan to better control asthma symptoms. FIG. 33H is another exemplary GUI 3380 displayed in response to the user-entered information in the user interface workflow relating to an alert. In this example, GUI 3380 may provide a reminder or suggestion to the user to consult the patient's medical team (e.g., to see if the medical team recommends a change in medical based on the patient's symptoms).

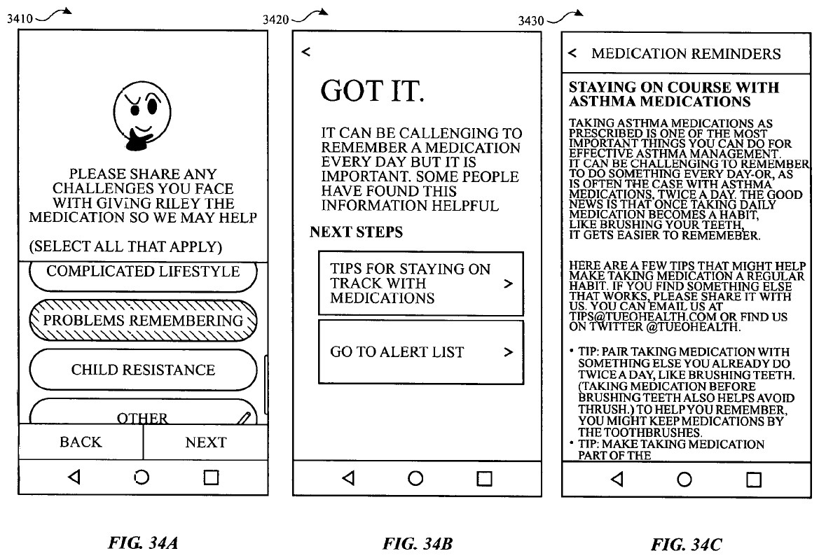

FIGS. 34A-34C are exemplary GUIs forming part of an interactive user workflow in which the user is provided with information that may help the user better control the patient's symptoms of a medical condition. In this example, FIGS. 34A-34C investigate reasons that a patient may be experiencing difficulty in complying with a medication treatment plan for alleviating symptoms. Specifically, FIG. 34A is an exemplary GUI 3410 that displays one or more selectable icons, each corresponding to a possible reason that a patient is finding it challenging to comply with medication. GUI 3410 prompts the user to select at least one reason. In response to the user selection, additional information may be displayed to help the user address and mitigate the selected reason. For example, in response to a user selection in GUI 3410 indicating that the user (e.g., a caregiver such as a parent) has problem remembering to give medication to the patient, a GUI 3420 as shown in FIG. 34B may be displayed to provide encouragement and/or selectable access to additional tips on how to stay on track with medication. In this example, in response to a user selection requesting such helpful tips, a GUI 3430 as shown in FIG. 34C may display a list of suggestions that may aid the user in remembering to regularly provide medication to the patient (e.g., pairing medication dose with another suitable regularly-performed task, such as brushing teeth). It should be understood that in other variations, such tips may additionally or alternatively include audio, video, and/or links to websites with additional information, etc.

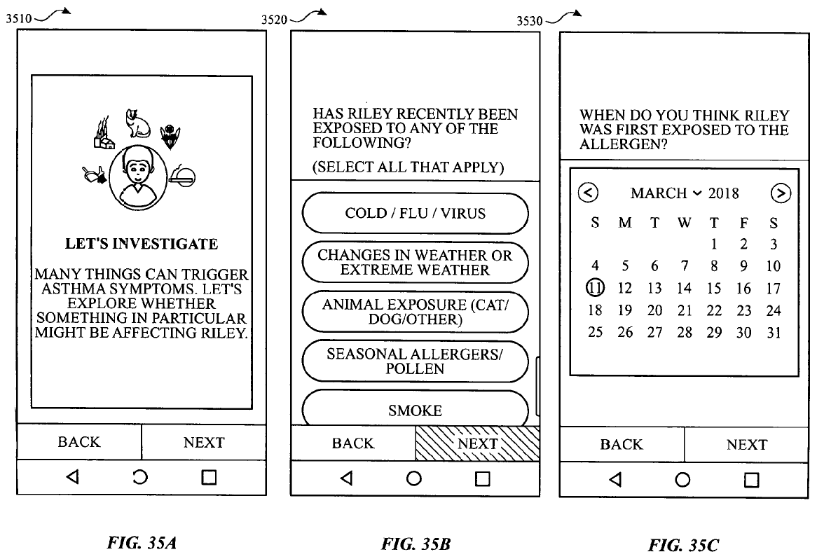

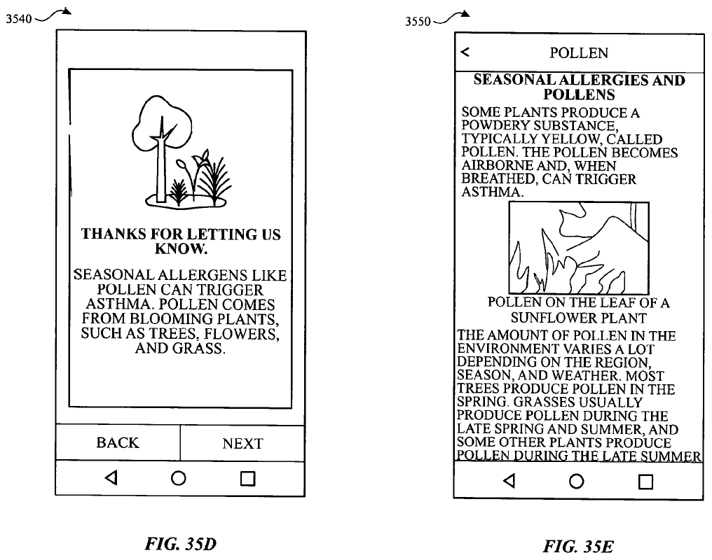

FIGS. 35A-35E are exemplary GUIs forming part of an interactive user workflow in which the user is provided with information that may help the user better control the patient's symptoms. Specifically, GUI 3510 shown in FIG. 35A may prompt the user to enter a user workflow to explore factors that may be triggering asthma symptoms experienced by the patient. In response to the user entering the workflow, a GUI 3520 as shown in FIG. 35B may be displayed. In this example for exploring triggers of asthma conditions, GUI 3520 may display a predetermined list of potential triggers (e.g., patient health conditions, environmental conditions) of asthma conditions. Each displayed trigger may be selected by the user. Exemplary triggers include, for example, a patient illness (e.g., cold, flu, other virus), weather changes or extreme weather, exposure to animals (e.g., cat, dog, other animal), seasonal allergens (e.g., pollen), and smoke, etc.

In response to a user selection of one or more triggers, additional follow-up questions may be displayed to prompt the user to provide additional information. For example, in response to a user selection in GUI 3520 that the patient was recently exposed to seasonal allergens, a GUI 3530 may prompt the user to enter an estimated earliest exposure date to the allergens, such as by displaying a calendar view with selectable days. In some variations, GUI 3530 may prompt the user to enter a respective estimated earliest exposure date for each potential trigger selected in GUI 3520. In other variations, GUI 3530 may prompt the user to enter an estimated exposure date collectively for all potential triggers selected in GUI 3520. In some variations, this trigger and/or exposure information may be stored as logged information for tracking purposes. For example, following entry of such information by a user, the trigger and/or exposure information may be displayed as logged information in a calendar view and/or list view in a tracking menu (e.g., similar to GUI 2700 shown in FIG. 27, or GUI 2900 shown in FIG. 29).

As shown in FIGS. 35D and 35E, exemplary GUIs 3540 and 3550 may provide educational information (which may include content similar to that of learning modules described above) regarding the one or more triggers selected in GUI 3520. For example, GUI 3540 may display a high-level summary of one or more selected triggers (e.g., seasonal allergens such as pollen) and their relationship to asthma. GUI 3540 may display an icon that is selectable to provide access to more detail information about the trigger. For example, as shown in FIG. 35E, the GUI 3550 may display an extended, detailed description of seasonal allergens such as pollen, and their effect on asthma conditions.

Although the above-described exemplary GUIs are primarily described and shown in the context of mobile applications (e.g., presented on a mobile device such as a mobile cellphone), it should be understood that other variations of the GUIs may be modified for display through an internet browser (e.g., on a desktop or laptop computer) and engagement with a user input device such as a mouse. Other variations may be modified for display on other suitable computer-based user interfaces.

### EXAMPLES

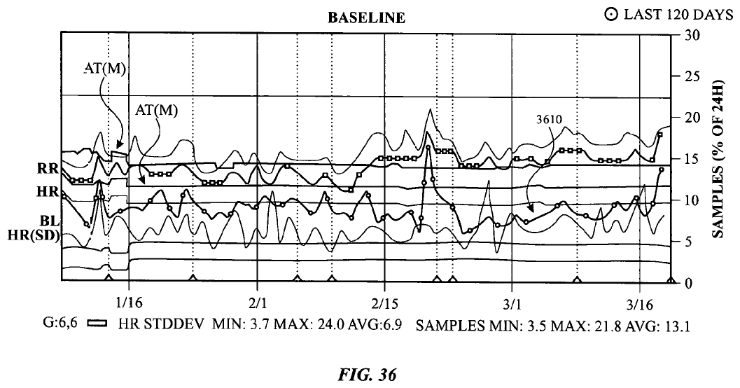

FIG. 36 is a plot depicting exemplary nocturnal patient data (e.g., derived parameters) over time. For example, the plot depicts median respiratory rate (RR) and median heartrate (HR) monitored over time. The respective interquartile ranges of RR and HR are also shown as shaded regions above and below the RR and HR lines. Standard deviation of heartrate (HR(SD)), which quantifies variance of heartrate during each night, is also shown. Additionally, the plots includes alert thresholds, including a minor alert threshold (AT(m)) and a major alert threshold (AT(M)) which may be used to trigger different alerts to a user. The alert lines are based on the standard deviation from a baseline (BL) over time. As shown in FIG. 36, the derived parameters, baseline, and alert thresholds vary over time. For example, FIG. 36 depicts average heart rate with line 3610 (each data point representing 50th percentile heart rate, or median, for that night), which rises slowly between about March 1 and March 14. This slow elevation in heart rate precedes a sudden increase in heart rate after about March 16.

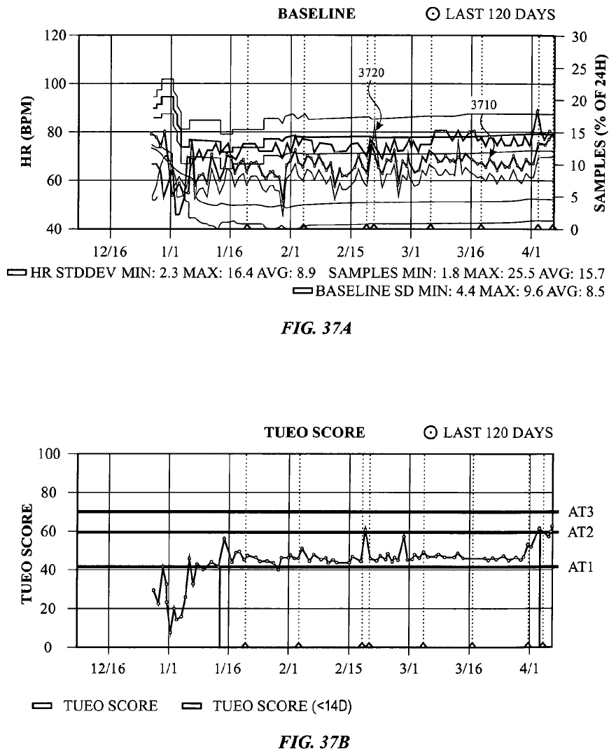

FIGS. 37A and 37B are plots depicting another set of exemplary nocturnal patient data over time. Specifically, FIG. 37A shows derived parameters similar to those shown in FIG. 36, including median heart rate depicted as line 3710 (each data point representing 50^{th} percentile heart rate for that night) and standard deviation of heart rate depicted as line 3720 (representing intra-night variability for that night). Over the course of about three months (January 1 to April 1), average heart rate increased slowly, before a larger increase at the time of an asthma event. Concurrently, change in heart rate standard deviation 3720 is seen to drop due to a loss of normal sleep variation in the heart rate.

FIG. 37A also illustrates values of major and minor alert thresholds AT(M) and AT(m) over time. An initial calibration phase occurred during the period between approximately December 27 through January 13. The alert thresholds AT(M) and AT(m) were higher during this initial calibration phase (e.g., to compensate for lower confidence in data calculated up to that point).

Although the present disclosure has been described in relation to various exemplary embodiments, various additional embodiments and alterations to the described embodiments are contemplated within the scope of the disclosure. Thus, no part of the foregoing description should be interpreted to limit the scope of the invention as set forth in the following claims. For all of the embodiments described above, the steps of the methods need not be performed sequentially.

What is claimed is:

1. A system to assist in the controlling of a patient's medical condition, comprising:
one or more sensors configured to sense individual patient data on or near a patient, wherein the individual patient data is related to at least one physiological parameter of the patient, wherein at least one of the sensors comprises a passive sensor that does not require patient activation or patient contact;
a transmitter configured to transmit the individual patient data to a processor; and
a processor configured to calculate a signal using the individual patient data and at least one of a baseline patient data or population data;
wherein the processor is further configured to calculate the signal using a combination of a proportional change, integral change and derivative change of the individual patient data and the at least one of the baseline patient data or population data.

2. The system of claim 1, wherein the individual patient data is derived individual patient data.

3. The system of claim 1, wherein the processor is further configured to generate the combination of the proportional change, integral change and derivative change from a combination of individual and non-individual data.

4. The system of claim 3, wherein the combination of individual and non-individual data utilizes a combination including at least two of the following: physiologic, home, local environment, and medical health records.

5. The system of claim 1, wherein the processor is further configured to generate an alert to the patient or a care provider of the patient.

6. The system of claim 5, wherein the alerts includes at least one of a medication change, avoidance of regional trigger, behavioral change, environmental change, education.

7. The system of claim 1, further comprising at least two sensors and wherein the processor is further configured to distinguish individual patient between different people in a bed.

8. The system of claim 1, wherein the processor is further configured to receive a response from the patient or a caregiver and to automatically adjust the status signal.

9. The system of claim 1, wherein the processor is further configured to calculate an inspiratory time and an expiratory time using the one or more sensors.

10. The system of claim 1, wherein the processor is further configured to calculate an I/E ratio using the one or more sensors.

11. A method to assist in the controlling of a patient's medical condition comprising:
using one or more sensors configured to sense individual patient data on or near a patient, wherein the individual patient data is sensing at least one physiological parameter of the patient, wherein at least one of the sensors comprises a passive sensor that does not require patient activation or patient contact;
transmitting the individual patient data to a processor; and
calculating a signal with the processor using the individual patient data and at least one of a baseline patient data and population data; and using a combination of a proportional change, integral change and derivative change in the individual patient data and the at least one of the baseline patient data and population data.

12. The method of claim 11, wherein the individual patient data is derived individual patient data.

13. The method of claim 11, wherein the baseline patient data is generated from patient medical health records.

14. The method of claim 11, wherein calculating the signal further utilizes home or local environment data.

15. The method of claim 11, further comprising generating an alert to the patient or a care provider of the patient.

16. The method of claim 11, wherein the alerts includes at least one of: a medication change, an avoidance of regional trigger, a behavioral change, an environmental change, and patient education information.

17. The method of claim 11, further comprising:
calculating at least one of an inspiratory duration, an expiratory duration, and an I/E ratio using the one or more sensors; and
comparing the at least one of the inspiratory duration, the expiratory duration, and the I/E ratio to a corresponding the inspiratory duration, the expiratory duration, and the I/E ratio to a corresponding threshold value.

18. The method of claim 17, wherein the corresponding threshold value is an absolute value.

19. The method of claim 17, wherein the corresponding threshold value is a patient-specific value derived from the at least one physiological parameter.

20. The method of claim 17, further comprising calculating the I/E ratio from a signal received from the passive sensor.

21. The method of claim 17, wherein the at least one of the inspiratory duration, the expiratory duration, and the I/E ratio is calculated using beat-to-beat variations in the heart activity detected by the one or more sensors.
